(19) United States
(12) Patent Application Publication
(10) Pub. No.: US 2021/0280287 A1
(43) Pub. Date: Sep. 9, 2021

(54) CAPACITY OPTIMIZATION ACROSS DISTRIBUTED MANUFACTURING SYSTEMS

(71) Applicant: VINETI INC., San Francisco, CA (US)

(72) Inventors: KHURRAM MAHMOOD, SAN RAMON, CA (US); STUART ALTMAN, EL CERRITO, CA (US); RICHARD BREWSTER WICKERSHAM, III, SAN FRANCISCO, CA (US); ERIC MATTHEW BELL, EMERYVILLE, CA (US); APAVEN STEPANYAN, Yerevan (AM); ANAND KOTHARI, CUPERTINO, CA (US); DAVIT DEMIRKHANYAN, Yerevan (AM); Roman Vladimirovich Kolesnev, Yerevan (AM); Armaan Rai, San Francisco, CA (US); VAHAN NERSISYAN, ABOVYAN (AM)

(21) Appl. No.: 17/182,429

(22) Filed: Feb. 23, 2021

Related U.S. Application Data
(63) Continuation-in-part of application No. 16/698,553, filed on Nov. 27, 2019.
Application No. 16/698,553 is a continuation-in-part of application No. 16/729,410, filed on Dec. 29, 2019.

(30) Foreign Application Priority Data
Jun. 23, 2020 (AM) 810

(51) Int. Cl. G16H 20/10; G16H 50/20; G16H 10/60; G06F 9/48; G06F 9/38

(52) U.S. Cl. CPC G16H 20/10; G16H 50/20; G06F 9/3838; G06F 9/4881; G16H 10/60

(57) ABSTRACT

The subject disclosure relates to systems, devices, and methods for executing operations related to executing a series of dependent scheduling operations associated with an individualized medicine supply chain and distributed scheduling systems. Also disclosed are system, method, and device embodiments for optimizing capacity of executable operations associated with producing an individualized therapeutic medicine.

### CROSS-REFERENCE TO RELATED APPLICATIONS

This application is a continuation-in-part patent that claims priority to U.S. patent application Ser. No. 16/698,553 titled, “CENTRALIZED AND DECENTRALIZED INDIVIDUALIZED MEDICINE PLATFORM”, filed Nov. 27, 2019 which claims priority to U.S. Provisional Patent Application No. 62/772,955 titled, “CENTRALIZED AND DECENTRALIZED INDIVIDUALIZED MEDICINE PLATFORM”, filed on Nov. 29, 2018. This application is also a continuation-in-part patent that claims priority to U.S. patent application Ser. No. 16/729,410 titled, “SMART LABEL DEVICES, SYSTEMS, AND METHODS” filed on Dec. 29, 2019 which claims priority to U.S. Provisional Patent Application No. 62/807,751 titled, “SMART LABEL DEVICES, SYSTEMS, AND METHODS” filed on Feb. 20, 2019. This application also claims priority to Armenian patent application number 810 filed on Jun. 23, 2020 and titled “CAPACITY OPTIMIZATION ACROSS DISTRIBUTED MANUFACTURING SYSTEMS”.

The entirety of the disclosures of the aforementioned applications are considered part of, and is incorporated by reference in, the disclosure of this application.

### BACKGROUND

The recent emergence in disease prevention and treatment known as precision medicine offers several benefits to patients. This approach to medicine allows for the provisioning of customized therapies to patients based on calculated predictions of which treatments may work best for a particular patient. For instance, CAR-T cell therapy is a cancer treatment that involves the extraction of T-cells from a patient's blood and modifying the cell surface to include chimeric antigen receptors that facilitate the production of a chemical to attack tumor cells. Although, these treatments are revolutionizing disease treatment, a complex chain of processes must be undertaken in order to generate the therapeutics ultimately administered to the patient.

For instance, autologous CAR-T process begins with apheresis through which one or more blood samples from a patient are collected, which are sometimes transported to storage facilities before arriving at a manufacturing facility. Once the patient-tailored therapeutic is manufactured, the therapeutic is transported back to a facility employing staff that can infuse the therapeutic medicine (e.g., manufactured CAR-T cells) into the patient. This generalized overview of various points along a personalized medicine supply chain is fraught with logistical challenges, scheduling challenges, stakeholder coordination challenges, monitoring and tracking challenges, and other related problems.

In one aspect, with respect to scheduling activities related to the manufacture of individualized therapies, materials have to be transferred from a collection site to a manufacturing site in a timely fashion to satisfy manufacturing requirements for a successful outcome. For instance, with respect to autologous cell and gene therapy manufacturing, a raw material (e.g., blood or tumor sample) is collected from a patient (e.g., at a collection site) but has to be transferred to a manufacturing site to undergo a multi-step manufacturing process resulting in the creation of the cell and gene therapeutic medicine. The manufactured therapeutic medicine must be scheduled for delivery to an infusion site such that the infusion of the medicine into a patient occurs in a timely manner.

In such an instance, several scheduling challenges exist. In an instance, a patient needs to be scheduled for collection if a sample based on an occurrence of several conditions, such as an availability of a scheduled collection slot at a collection site that coordinates with a patient schedule. Furthermore, adequate resources need be available at the scheduled time, such as the availability of qualified providers to perform the collection activities. Also, the scheduling of the sample collection can only occur where a courier is scheduled to pick up the sample from the collection site and drop off the sample at the next facility such as a manufacturing site or intermediate storage facility.

Furthermore, identifying a scheduling date and time not only depends on the above factors, but also on the estimated duration of activities (e.g., time for collecting the sample) and such duration can vary based on additional factors such as the patient (e.g., health conditions, special needs, etc.) and the type of collection (e.g., tumor sample collection) and therapy. For instance, during a tumor sample collection, the actual duration of collection is unknown a-priori. As such, there are several levels of complexity required for consideration in order to effectuate an efficacious scheduling regime. Other areas of complexity include the consideration of courier scheduling dependencies (e.g., courier availability and efficiency in a particular region and with respect to a particular route), manufacturing site dependencies (e.g., availability to manufacture a particular product during a particular time slot in which the collected material arrives, existence of manufacturing capacity and particular raw materials such as AAV vectors as well as availability of qualified technicians to manufacture the product), and infusion site dependencies (e.g., patient condition and availability when product arrives at infusion site, physician and qualified staff availability, resource availability, etc.). Accordingly, several scheduling challenges exist.

In other respects, scheduling challenges also include the actual changes to scheduling that occur at respective times. For instance, patients may have a deteriorated condition on a scheduled date for collection or infusion or a material collected from a patient may not possess the desired characteristics for manufacturing a therapeutic product. Other variability can exist with respect to couriers (e.g., couriers are late for pick-up or drop off, transportation delays occur such as due to traffic congestion or weather conditions, etc.) and manufacturing capacity (e.g., down-stream delays in manufacturing can result from delays in preceding steps such as collection or courier delays, delays may occur due to manufacturing machine malfunctions or unavailability of resources required for manufacturing). As such, schedules often need to be changed or adjusted and current scheduling techniques are antiquated and are unable to accommodate such volatility in scheduling.

As an example, existing scheduling technologies in the personalized medicine industry are inflexible and fail to capture accurate schedules given that such schedules are based on pre-configured, hard-coded, and inaccurate assumptions (e.g., pre-configured time duration of a journey from site A to site B) for the performance of various tasks such as a fixed time assumption for transportation of a material from one site to another site. Furthermore, such scheduling technologies are inept at accounting for the complexities associated with scheduling in the personalized medicine industry and merely rely on manual processes that result in faulty schedules, create a plethora of scheduling conflicts, errors, inefficient scheduling, forgone manufacturing capacity, inefficient labor and resource allocation, delay in and inability to respond to sudden schedule changes and an absence of notification between relevant stakeholders. As such, challenges associated with scheduling technologies related to the personalized medicine industry are pervasive and unsolved. Given the rise of personalized medicine supply chains and the existence of several challenges associated with such supply chains, there is a need for new technologies to solve these problems.

### SUMMARY

The following presents a summary to provide a basic understanding of one or more embodiments of the invention. This summary is not intended to identify key or critical elements or delineate any scope of the particular embodiments or any scope of the claims. Its sole purpose is to present concepts in a simplified form as a prelude to the more detailed description that is presented later. In one or more embodiments, described herein are systems, devices, apparatuses, computer program products and/or computer-implemented methods that facilitate an interaction between an individualized medicine system and distributed scheduling systems.

According to an embodiment, a computer-implemented method is provided. In an aspect, the method can include accessing, via a client device, a distributed scheduling system. The method also includes transmitting, using the client device, a query analysis to the distributed scheduling system to perform a scheduling operation. Furthermore, in another aspect, the method includes receiving, from the distributed scheduling system, a dynamic schedule analysis, including one or more insights associated with the trigger event. In yet another aspect, the method provides for outputting, via a display module, a scheduling interface effective to render content associated with the scheduling operation; and actionable controls associated with augmentation of the scheduling operation.

### BRIEF DESCRIPTION OF THE DRAWINGS

FIG. 1A illustrates an example, non-limiting diagram of a representative environment 100A in which a distributed scheduling system associated with a personalized medicine supply chain can be utilized in accordance with one or more implementations described herein.

FIG. 1B illustrates an example, non-limiting block diagram of a representative environment 100B in which a distributed scheduling system associated with a personalized medicine supply chain and a blockchain system can be utilized in accordance with one or more implementations described herein.

FIG. 2 illustrates an example, non-limiting diagram of a representative environment 200 in which cloud-based services can be used to provide features corresponding to the automated generation of scheduling and capacity optimization operations associated with a distributed scheduling system in accordance with one or more implementations described herein.

FIG. 3A illustrates an example, non-limiting distributed scheduling system 300A and an example distributed scheduling engine module in accordance with one or more implementations described herein.

FIG. 3B illustrates an example, non-limiting flow diagram 300B of optimizing capacity and scheduling amongst a distributed scheduling and capacity providers corresponding to a personalized medicine supply chain in accordance with one or more implementations described herein.

FIG. 3C illustrates an example, non-limiting diagram of an interactive scheduling interface 300C rendered by distributed scheduling system at a client device in association with a personalized medicine supply chain in accordance with one or more implementations described herein.

FIG. 3D illustrates an example, non-limiting flow diagram 300D of scheduling and capacity reservation routines associated with the distributed scheduling system in accordance with one or more implementations described herein.

FIG. 4A illustrates an example, non-limiting distributed scheduling system 400A and an example rescheduling engine module in accordance with one or more implementations described herein.

FIG. 4B illustrates a flow diagram of an example, non-limiting quality control operations and estimation operations associated with the distributed scheduling system in accordance with one or more implementations described herein.

FIG. 5 illustrates an example, non-limiting distributed scheduling system 500 and an example capacity engine module in accordance with one or more implementations described herein.

FIG. 6 illustrates an example, non-limiting distributed scheduling system 500 and an example load balancing engine module in accordance with one or more implementations described herein.

FIG. 7 illustrates an example, non-limiting distributed scheduling system 500 and an example prediction engine module in accordance with one or more implementations described herein.

FIG. 8 illustrates a flow diagram of accessing, via a client device, a distributed scheduling system in accordance with one or more implementations described herein.

FIG. 9 illustrates a flow diagram of querying, via a client device, a distributed scheduling system in accordance with one or more implementations described herein.

FIG. 10 illustrates a flow diagram of generating, via a distributed scheduling system a schedule record in accordance with one or more implementations described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section. One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Techniques described herein provide cloud-based platform technologies that allow for the execution of a range of operations related to transactions that occur as part of an individualized medicine supply chain. Various implementations receive data from multiple data sources, identify attributes of the data, and execute an operation to facilitate a stakeholder activity in connection with the personalized medicine supply chain. For instance, in a non-limiting aspect, techniques described herein provide for the coordination of supply chain events (e.g., automated courier scheduling, raw material delivery, kit tracking capabilities, raw and final product preparation, streamlining ordering and scheduling, capacity and resource management, and several other activities).

Furthermore, systems, devices, methods, environmental and architectural embodiments disclosed herein disclose one or more platform technology that facilitates a multi-tenant deployment of managing platform identity information, facilitating regional separation capabilities to support data residency requirements of the system, and/or facilitates satisfying separation requirements between controlled data set users and uncontrolled data set users. In other implementations, embodiments disclosed herein include distributed scheduling systems that execute distributed scheduling operations in connection with capacity subsystems and operational constraints associated with a personalized medicine supply chain. In an aspect, an example environment 100A is disclosed in which various aspects described herein can be employed.

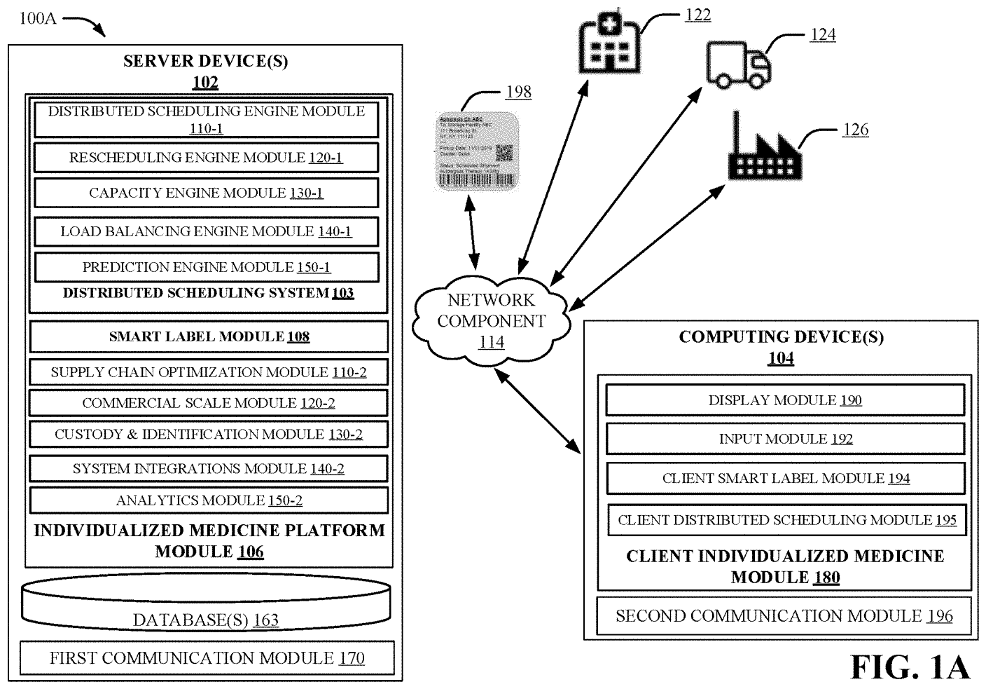

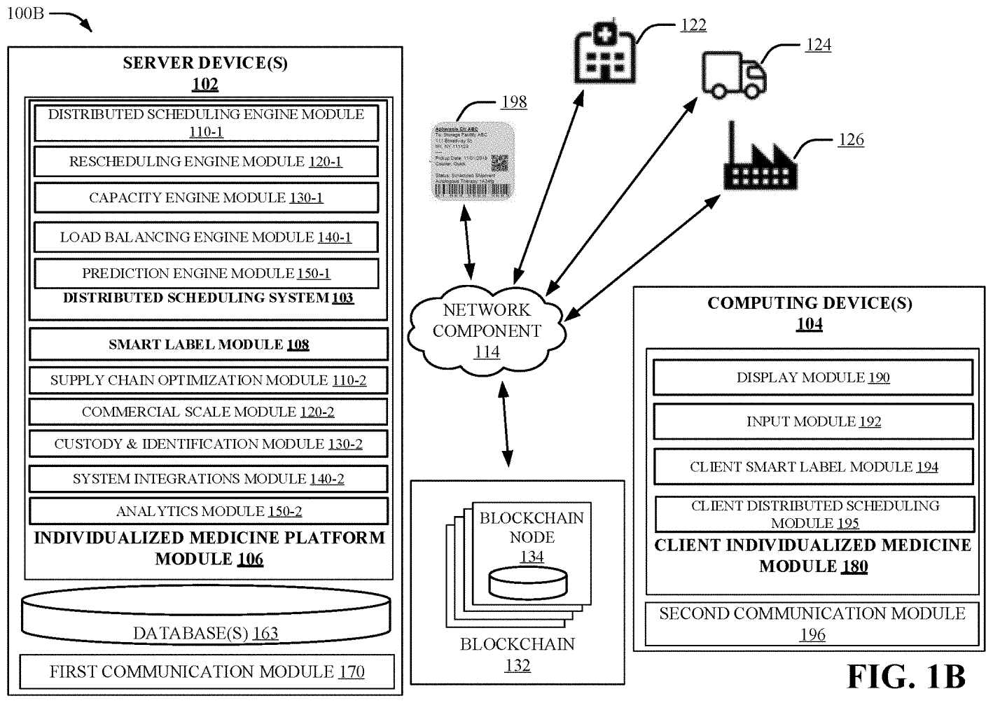

FIG. 1A illustrates an example, non-limiting diagram of a representative environment 100A in which a distributed scheduling system associated with a personalized medicine supply chain can be utilized in accordance with one or more implementations described herein.

In an aspect, environment 100A can include one or more server device(s) 102 and one or more computing device(s) 104 that can generate, curate, track, monitor and store data associated with transactional processes of an individualized medicine supply chain. For instance, environment 100A can facilitate a tracking of events associated with manufacturing gene therapies and cell therapies for target patients. In an aspect, computing device(s) 104 can be a desktop computing device, smart phone, tablet, laptop, smart watch, and other suitable type of computing device.

In an aspect, the terminology “individualized medicine supply chain” or “personalized medicine supply chain” and other variations are used to denote information that is generated from a combination of activities, events, materials, and devices utilized by various stakeholders to procure a personalized medicine therapeutic for a patient. In an aspect, an individualized medicine supply chain can begin with a decision to treat a patient with a personalized therapy (e.g., a therapeutic medicine manufactured from a patient's own biological materials such as cells) to the manufacturing of a personalized therapeutic medicine and finally to a point of infusing the personalized therapeutic medicine into a patient vein. For example, server device(s) 102 can acquire data, process the data to curate the acquired data, and generate queries for application to the data to perform various analytics, generate reports, track a chain of identity of various materials, track a chain of custody associated with various materials, and execute other such operations. In another aspect, server device(s) 102 can include individualized medicine platform module 106 that can employ various operations in connection with supply chain optimization module 110-2, commercial scale module 120-2, custody & identification module 130-2, system integrations module 140-2, analytics module 150-2, and other such modules.

In an aspect, individualized medicine platform module 106 can perform a range of operations related to data corresponding to individualized supply chain events such as point-of-care collection (e.g., collection of samples that meet threshold quality standard requirements), tracking supply chain events (e.g., as relates to manufacturing a personalized therapeutic for a patient), optimizing resources (e.g., fulfilling manufacturing orders), security and compliance (e.g., ensuring data quality, data validation, and data encryption mechanisms are implemented), supply chain orchestration events (e.g., scheduling couriers, raw material delivery, tracking kits and materials and raw materials), ordering and scheduling activities (e.g., scheduling of sample collections, manufacturing, administration of medicine, etc.), reporting tasks (e.g., reporting quality findings, compliance activities, chain of identity information, chain of custody information, etc.), and/or facilitating system integrations (e.g., data integrations, data modifications, insight procurement, etc.).

In an aspect, individualized medicine platform can employ supply chain optimization module 110-2-2, commercial scale module 120-2, custody & identification module 130-2, system integrations module 140-2, and analytics module 150-2 to perform a range of operations (e.g., by providing instructions for execution by a processor of server device(s) 102). In an aspect, individualized medicine platform module 106 can employ supply chain optimization module 110-2 to execute ordering operations, scheduling operations, and resource planning operations. Furthermore, supply chain optimization module 110-2 can execute tasks that orchestrate logistical events (e.g., coordinate couriers) and perform data (e.g., representing the movement of materials and/or products through the individualized medicine supply chain) tracking tasks to allow for transparent visibility of activities performed along the individualized medicine supply chain. For instance, supply chain optimization module 110-2 can execute scheduling operations corresponding to pickup and delivery of raw materials, final products, biological materials, and other such items by stakeholders (e.g., couriers, third party logistics providers, etc.) to create efficiencies in transportation of materials.

In an aspect, individualized medicine platform module 106 can execute supply chain optimization module 110-2 to facilitate ordering one or more therapy for a patient. For instance, supply chain optimization module 110-2 can intake one or more indication data sets, therapy descriptive data sets, patient information (e.g., name, date of birth), study detail data (e.g., if a patient is affiliated with a study), patient consent data, order cancellation data, and other such information. Furthermore, supply chain optimization module 110-2 can facilitate a selection of a treatment ordering site (e.g., center of excellence) and treatment prescriber (e.g., physician data). In a non-limiting embodiment, supply chain optimization module 110-2 can employ a set of rules and requirements to limit selection options based on intake data as applied against a set of ordering rules customized to a particular set of circumstances (e.g., patient conditions, capacity conditions, delivery conditions, etc.).

In another aspect, supply chain optimization module 110-2 can execute scheduling operations. For instance, supply chain optimization module 110-2 can intake data corresponding to scheduling a therapeutic order such as collection site data, contact data, delivery site data, calendar scheduling data, and other such data. In a non-limiting embodiment, supply chain optimization module 110-2 can employ a set of rules and requirements applied against intake data to determine a limited selection of options allowable for scheduling. For instance, one or more rules can correspond to existing courier scheduling dates, therapy-specific conditions for transportation and delivery time constraints, logistical restrictions, and other such requirements.

In an aspect, machine learning techniques can be employed to group such intake data and evaluate data patterns related to ordering and scheduling. Furthermore, in some non-limiting embodiments, machine learning operations can be employed to determine predictive likelihoods and/or predictive efficiencies to be achieved from selecting a combination of ordering and selection related to a therapy, region, user, and other such platform attributes. In another aspect, various constraints, requirements, and rules can be applied against intake data by supply chain optimization module 110-2 in order to effectuate occurrence of timely ordering and scheduling events. For instance, for fresh blood-based therapies, once collected, patient blood has shelf-lives and expiration dates after which the material is no longer usable to manufacture therapeutic products. For cryopreserved material, the shelf-life is longer. However, the effects of cryopreservation on the collected cells are still not fully understood and it introduces further complexity due to freezing and then thawing of material. Accordingly, individualized medicine platform module 106 employs rules and conditions to ensure the material has an efficacious transition through the supply chain up through infusion as a finished therapeutic product into a patient.

In another aspect, supply chain optimization module 110-2 can execute (e.g., using a processor) product tracking operations to facilitate transparency as supply chain events occur. For instance, supply chain optimization module 110-2 can acquire, curate, analyze, integrate, and/or generate data associated with a material workflow through various stages of a supply chain. As a non-limiting example, supply chain optimization module 110-2 can receive scheduling data (e.g., representing instructions to execute a scheduling task) from client individualized medicine module 180 (e.g., receiving data from input module 192), where the scheduling data represents the event of collecting blood samples from a center of excellence (e.g., hospital qualified for a respective therapy). Furthermore, supply chain optimization module 110-2 can receive data (e.g., from a sensor incorporated into the kit, from courier data confirming delivery of the kit, from a smart label attached to the kit, etc.) associated with the delivery of a kit to the center of excellence to facilitate the extraction of the blood samples (e.g., at a point of care setting) and submission of such sample within a kit. In another aspect, supply chain optimization module 110-2 can receive collection data representing a confirmation that the kit with the blood sample has been picked up by a courier. In an aspect, supply chain optimization module 110-2 can further receive data indicating cells (within the blood) have been shipped (in the process of being transported) to a manufacturing facility.

In a non-limiting embodiment, supply chain optimization module 110-2 can execute collection (e.g., blood collection) operations. For instance, supply chain optimization module 100 can intake data corresponding to scheduling a collection of blood such as patient (blood) test result data, patient collection bag label data, patient identity data, collection bag identification data (e.g., unique donation number, apheresis ID, site data, etc.), patient data (e.g., weight data, CBC count), bag detail data (e.g., amount of blood collected (mL), anticoagulant type, anticoagulant volume, collection start time/end time, change of custody confirmation data (e.g., bag identifier label or scanned bag data), collection bag shipping data, and other such data. In a non-limiting embodiment, supply chain optimization module 110-2 can employ a set of rules and requirements to limit selection options based on intake data as applied against a set of collection rules customized to a particular set of circumstances (e.g., type of therapy, patient conditions, capacity conditions, delivery conditions, collection facility scheduling constraints, etc.).

In other aspects, supply chain optimization module 110-2 can generate and/or receive tracking data associated with events along the manufacturing portion of the supply chain (e.g., track and confirm that a courier shipment has been received, quality inspection occurred and results are compared to a threshold target result, manufacturing of personalized therapeutic has commenced, quality inspection occurred and results were compared to a threshold target result, finished product has shipped, etc.) and the patient administration portion of the supply chain (e.g., tracking of therapeutic until receipt by center of excellence, scheduling of infusion of therapeutic, infusion occurred, long-term tracking of patient outcome, etc.).

In a non-limiting embodiment, supply chain optimization module 110-2 can execute satellite manufacturing (e.g., personalized therapies) operations. For instance, supply chain optimization module 100 can intake data corresponding to scheduling a satellite manufacturing operation. For instance, supply chain optimization module 110-2 can intake data such as therapy data, study number data (if applicable), Chain of Identity (COI) information, shipment verification data, collection data, receiving documentation data (e.g., is the raw material kit shipped properly such as inside a specialized pouch, was the raw material stored at required temperatures such as temperatures to preserve a vapor phase of liquid nitrogen as per some material requirements, missing items, mismatching numbers, damages, etc.), shipment verification data, number of (blood) bag data, print label data, confirmation data (e.g., manufacturer labels applied to bags), COI identifier scan data on collection bag and/or cassette (if applicable), cryopreservation data (e.g., product volume data, mononuclear cell count data, cell viability data, summary data, bag shipment data, quality release data, document release data, transfer data, authorized user signature data, shipping checklist data, and other such data. In a non-limiting embodiment, supply chain optimization module 110-2 can employ a set of rules and requirements to limit selection options based on intake data as applied against a set of satellite manufacturing rules customized to a particular set of circumstances (e.g., patient conditions, capacity constraints, delivery conditions, manufacturer facility scheduling constraints, etc.).

In another non-limiting embodiment, supply chain optimization module 110-2 can execute central manufacturing (e.g., personalized therapies) operations. For instance, supply chain optimization module 100 can intake data corresponding to scheduling a central manufacturing operation. For instance, supply chain optimization module 110-2 can intake data such as lot number data (e.g., manufacturing lot number for the therapeutic product), confirmation data, shipment condition data, shipment detail data, shipment COI identification data, air waybill data, shipment pouch data, first stimulation data (e.g., start date data), second stimulation data, harvesting data (e.g., start date data), final product data (e.g., number of bags of therapeutic after manufacturing process, product expiry date data, product label data (e.g., print ready label for bag of final product), label attachment confirmation data, respective bag number label application data, unused label disposal data, bag selection data (e.g., for shipment), quality release data, product transfer to shipper data, shipping checklist data, and other such intake data.

In a non-limiting embodiment, supply chain optimization module 110-2 can employ a set of rules and requirements to limit selection options based on intake data as applied against a set of central manufacturing rules customized to a particular set of circumstances (e.g., patient conditions, capacity constraints, delivery conditions, manufacturer facility scheduling constraints, etc.). In an aspect, supply chain optimization module 110-2 can provide scheduling options based on manufacturing capacity data. For instance, a set of scheduling date options can be provided for scheduling manufacturing based on manufacturing capacity data queried by supply chain optimization module 110-2.

As such, various system components such as the scheduler component and manufacturing capacity component can be used to satisfy requirement criteria to facilitate a system determination (e.g., date availabilities for manufacturing occur). In another instance, a capacity-based scheduling operations employed by supply chain optimization module 110-2 can utilize scheduling rules that correspond to a number of manufacturing slots available per week, number of slots currently scheduled, additional bookings available for the week, and generate determinations (e.g., using supply chain optimization module 110-2) as to dates and times available for manufacturing. Furthermore, capacity-based scheduling operations can include supply chain constraints and rules such as number of manufacturing legs required, number of shipping legs required for product to arrive, time for shipment, and other such considerations.

In an aspect, scheduler component and manufacturing capacity component can provide a normalized view of manufacturing capacity across distributed manufacturing sites (e.g., sites owned by pharma companies, independent contract manufacturing organization (CMO) sites). Furthermore, manufacturing capacity component can access data from multiple data stores to allow scheduler component to provide a unified view of manufacturing capacity by therapy, region, site, any arbitrary tag applied to a site or other segment to ascertain capacity capabilities and constraints. Furthermore, in a non-limiting embodiment, manufacturing site capacity data can be stored in a registry at server device(s) 102 (e.g., database(s) 160) in order to accommodate data from a variety of manufacturing site sources (e.g., data sourced via integrations such as Manufacturing Execution Systems (MES) systems).

In an example non-limiting instance, a healthcare provider (HCP) responsible for administering particular therapies can execute an order for a therapy (e.g., therapy permitted to order) associated with a customer instance at individualized medicine platform module 106. Furthermore, scheduling component can determine capacity data from distributed center of excellence capacity registry data store that comprises distributed apheresis site capacity data and infusion site capacity data (e.g., unreserved capacity and contractually reserved capacity for therapies). Furthermore, scheduler component can employ manufacturing capacity component to access data from customer specific capacity registries associated with distributed manufacturing sites published capacity data to determine manufacturing site capacity capabilities. As such, manufacturing capacity data can be based on customer configuration information and scheduling rule requirements. Furthermore, scheduling component can employ predictive determination components that predict manufacturing times (e.g., based on machine learning analysis of historical and current data), optimize scheduling activities, and determine waitlist and over-scheduling decisions based on site specific rescheduling data and rules.

In other aspects, supply chain optimization module 110-2 can also execute operations based on intake of distribution center (e.g., global distribution) intake data (e.g., specimen status upon receipt, shipment verification data, COI data, condition of shipment data, etc.) and order status tracking intake data or presentation data (e.g., shipping status, satellite lab status, manufacturing status, distribution center status, infusion status, etc.). For instance, the collection stage can be presented (e.g., using display module 190) at a user interface with stage data, phase data (e.g., test result stage), shipping stage data (e.g., pending, shipped, complete, etc.), site data (e.g., center of excellence, cryogenic laboratory, test institution, distribution center, hospital, etc.), timeline data, and other such information.

In yet another aspect, supply chain optimization module 110-2 can also execute operations such as cancellation of approved orders (e.g., intake order cancellation request data, etc.), cancellation of in-progress or submitted orders (e.g., patient consent data, order cancellation request data, etc.), rescheduling of tasks (e.g., collection, manufacturing, infusion, etc.), intake of infusion intake data (e.g., product and/or shipment receipt data, transfer product to storage data, condition of shipment data, real-time tracking data, real-time alert notification data, etc.), user management operations (e.g., creation of a user, etc.), change request operations, and other such operations. In an aspect, each operation requires authentication of one or more user and each operation can be performed by different authorized users and user devices corresponding to predefined permissions.

For instance, a first user (e.g., nurse, staff member) at a first site (e.g., center of excellence, hospital, etc.) may be assigned permissions to enroll patients and place orders for a patient therapy, a second user (e.g., patient operations or central scheduler user) at a second site (e.g., manufacturing facility) may be assigned permissions to access enrollment documentation (e.g., via individualized medicine platform module 106) and initiate transmission of a therapeutic order to a third user (e.g., logistics personnel) at a second site (e.g., manufacturing facility) with permissions to approve a therapy order and schedule a shipment of the therapy. Furthermore, the second user may be assigned permissions to send product data to first user. In such instance, each user device is assigned roles having permissions and limitations to facilitate execution of respective operations using individualized medicine platform module 106.

In an aspect, computing device 104 can employ display module 190 to transform the data to various types of output information (e.g., text, charts, video, audio, graphs, tables, and other such output) that are based upon order data, schedule data, capacity data, patient identification data, sample identification data, courier data, manufacturer data (e.g., batch number), electronic record data, milestone data associated with medicine manufacturing, medicine quality data (e.g., cell count data), notification data, storage data (e.g., cryogenic storage of sample), patient treatment data, patient outcome data (e.g., long-term monitoring), payment data (e.g., reimbursement information), assay quality control data, sample storage data (e.g., temperature data of a storage facility received from temperature sensors, orientation or tilt data of a package containing patient samples or therapeutic medicines, humidity data of a manufacturing environment, etc.), monitoring data (e.g., information corresponding to sensors such as GPS sensors employed in factories, warehouses, laboratories, pharmacies, etc.), financial data, inventory data, and other such data.

In another aspect, supply chain optimization module 110-2 can generate, receive, curate, and/or transform ordering data, scheduling data, and/or resource management data. In an aspect, ordering data can represent instructions to order raw materials, patient samples, intermediary materials, final products (e.g., therapeutic drugs), and other such items associated with the individualized medicine supply chain. In an aspect, several stakeholders (e.g., manufacturers, physicians, patients, etc.) can execute ordering tasks based on authorization data representing permission credentials to perform an order. Furthermore, in an aspect, supply chain optimization module 110-2 can generate, receive, curate, and/or transform scheduling data to facilitate a coordination of collection (e.g., by nurses, couriers, manufacturers, etc.), manufacturing, and treatment administration of materials and final products. As such, supply chain optimization module 110-2 can facilitate the streamlining of scheduling and coordination activities. In yet another aspect, supply chain optimization module 110-2 can perform resource management tasks to optimize manufacturing capacities to manufacturing needs of patient and efficiently coordinate the fulfillment of orders for final products, raw materials, and/or samples.

In another non-limiting embodiment, individualized medicine platform module 106 can employ commercial scale module 120-2 (for execution by a processor of server device(s) 102) to perform operations such as acquiring and curating data associated with the manufacturing of personalized medicines, training of various stakeholders (e.g., onboarding, training and collaboration activities of healthcare providers), and configuration tasks (e.g., configuring modules and functions of the platform to perform client specific activities such as clinical activities, etc.). For instance, commercial scale module 120-2 can perform operations and functions to facilitate a determination as to whether manufacturing events or activities comply with manufacturing and compliance standards starting with point of care activities through manufacturing and final product delivery activities. As a non-limiting example, commercial scale module 120-2 can execute operations to ensure that a patient's identity is confirmed and verified (e.g., compare driver's license data, signature data, biometrics such as finger prints or facial recognition, etc.), that labels are verified to contain correct information, and correct products have been shipped.

In another aspect, commercial scale module 120-2 can perform operations associated with payment reimbursement for the performance of activities associated with individualized therapies. For instance, commercial scale module 120-2 can acquire, generate, and curate data associated with triggering events related to the procurement and administration of individualized therapies for patients. In an aspect, such triggering events can correspond to requirements that satisfy payer data needs (e.g., for payment reimbursement to occur) and commercial operator data needs. In another aspect, commercial scale module 120-2 can receive payment data and transform such payment data in various formats and structures that can expedite automated payment mechanisms that satisfy payment process requirements (e.g., revenue recognition processes). Furthermore, in an aspect, commercial scale module 120-2 can monitor patient data and transform patient data over a long-term period of time.

In another non-limiting embodiment, individualized medicine platform module 106 can employ custody & identification module 130-2 (for execution by a processor of server device(s) 102) to perform operations such as acquiring, generating, curating, transforming, monitoring, and storing data associated with chain of identity and/or the chain of custody of various entities, materials, and product along the individualized medicine supply chain. For instance, custody & identification module 130-2 can perform operations (e.g., such as generating and storing location data associated with a sample) that can allow for the traceability of patient samples (e.g., blood, cells, etc.).

As a non-limiting example, chain of custody & identification module 130-2 can perform audit operations (e.g., curate data and generate data representing an audit trail of handling events of cell products, custodial events associated with any product or material, identification correspondence of any material or product, etc.), quality validation operations (e.g., perform data quality validation checks and process validation reviews of data), record generation operations (e.g., create a platform of record of all data to enable transparency and instill confidence in stakeholders, generate reports based on data, etc.), and perform security operations such as encrypting data and performing authorization and validation operations to ensure only authorized user profiles can access respective data sets and representative information. In an aspect, chain of custody & identification module 130-2 can acquire and curate a set of data that includes time data that an event occurred, event identifier (e.g., signature occurred, label scanned, etc.), signature data associated with the identity of an entity performing a task at such time, description data describing the event that occurred (e.g., verification of a patient, scanning a label, ensuring a label matched an identifier number, verifying apheresis occurred, handing off a raw material to another entity, etc.), location data, identity data of a user profile performing the event.

In an instance, a chain of custody (COC) event ca be tracked via a scanning of a label corresponding to a raw material (e.g., blood) from the time it leaves a patient body (e.g., collection), through its path throughout the supply chain (e.g., shipment, cryogenic storage, manufacturing, etc.) to infusion of the therapeutic (e.g., blood) into the patient body. In an aspect, custody & identification module 130-2 can intake chain of custody and chain of identity (COI) data from a label (e.g., on a blood bag) via a scanning mechanism or other intake form. As an example, a chain of custody event can occur upon a raw material arriving at a manufacturing facility and custody & identification module 130-2 can intake such COC events. In another instance, a raw material arriving at a cellular laboratory can present several COC events taken in by custody & identification module 130-2.

At day 1, cell processing data can be utilized by supply chain optimization module 110-2 and corresponding COI event data (based on a scanning of the blood bag label) can be generated by custody & identification module 130-2. Furthermore, upon the bag being labeled and readied for shipping, upon another scanning of the label, a COC event can be generated by custody & identification module 130-2. Likewise, a COI and COC event can be generated by custody & identification module 130-2 based upon an occurrence of a shipping event (e.g., location to location material transition), a manufacturing quality user evaluating the test results of a manufactured product, a manufacturing operator confirming an occurrence of COC event (e.g., person to person material transition), product moving from one machine to another during manufacturing and other such events. In an aspect, custody & identification module 130-2 can generate a data store of all login events capable of access via one or more query request initiated using client analytics module. Furthermore, custody & identification module 130-2 can generate a COI transaction log corresponding to all events coupled to a COI number. Also, custody & identification module 130-2 can generate a COC transaction log representing each occurrence of a hand-off of raw material and/or product throughout the supply chain.

In another non-limiting embodiment, individualized medicine platform module 106 can employ system integrations module 140-2 (for execution by a processor of server device(s) 102) to integrate data from other systems with data generated, acquired, curated, analyzed and stored within database 160 or other data stores of environment 100A. In an aspect, system integrations module 140-2 can perform system integration operations such as acquiring, curating and storing data extracted from courier systems, enterprise resource planning systems, customer relationship management systems, manufacturing execution systems (MES), laboratory information management systems (LIMS), electronic batch record system (EBR), and quality management system (QMS). In general, individualized medicine platform 106 can facilitate the coordination, orchestration, and performance of ordering and scheduling operations, collection operations (e.g., acquiring data as per GMP requirements), transportation operations (e.g., generating data related to logistics activities, creating efficiencies via logistics automation), manufacturing operations (e.g., managing and controlling the deployment of resources), delivery operations (e.g., generate product tracking data), and payment reimbursement operations (e.g., acquiring and evaluating data related to administration of therapeutic).

In a non-limiting embodiment, systems integration module 140-2 can integrate systems that perform operations in connection with individualized medicine platform module 106 operations via various integration mechanisms. As an example, an onboarding subsystem that executes patient and site (e.g., center of excellence, hospital, etc.) onboarding operations (e.g., hub and patient support systems) can integrate with supply chain optimization module 110-2 to facilitate center of excellence setup tasks and patient selection activities. Furthermore, integration module 140-2 can integrate supply chain optimization module 110-2 with an electronic health record (EHR) system or LIMS system to facilitate individualized medicine platform module 106 execute patient scheduling operations in connection with cell and tissue collection tasks and constraints accessed from the EHR and/or LIMS systems.

As another example, integration module 140-2 can integrate supply chain optimization module 110-2 and logistics systems to facilitate execution of scheduling, tracking, monitoring, and other logistics operations by individualized medicine platform module 106 based on shipping constraints (e.g., bookings, weather delays, etc.) provided by logistics systems. Accordingly, other integrations facilitated by integration module 140-2 can include individualized medicine platform module 106 integrating the individualized medicine platform module 106 manufacturing scheduling capabilities (e.g., scheduling he manufacturing slots) with enterprise resource planning (ERP) systems (e.g., manufacturer ERP's indicating manufacturing capacity constraint information), Chain of Identity (COI) and/or Chain of Custody (COC) recordation operations with IVIES systems (e.g., based on manufacturing traceability information), infusion operations with EHR and/or Hub systems (e.g., indicating product delivery information and constraints upon which infusion is based), payer reimbursement operations with reimbursement hub systems (e.g., indicating order-to-cash data), and/or ongoing monitoring operations with Real World Evidence Systems (RWE) (e.g., to provide access to data regarding usage, benefits and risks of the therapy).

In another aspect, integration module 140-2 can employ a unified authentication system to execute authentication of an identity of a user (e.g., identity provider) utilizing individualized medicine platform module 106 and integrated systems (e.g., using integration module 140-2) such as a web application, a web service system, a storage service (e.g., data store, database, server, etc.) employed to perform a task in connection with the individualized medicine platform module 106. In an instance, an access management system or identity provider (e.g., employed across one or more server) can be configured to integrate (e.g., using integration module 14) with individualized platform module 106 and other systems in accordance with user (e.g., customer such as pharmaceutical company) criteria to act as an identity authentication mechanism to authenticate user identity and respective user roles (e.g., user permissions to execute a limited set of operations). In an aspect, the access management system can execute operations such as creation, maintenance and management of identity information of users and authentication operations for individualized medicine platform module 106 and integrated systems based on storage of user credential data.

In an aspect, an access management system in connection with integration module 140-2 can employ a single sign on (SSO) authentication mechanism configured to relate a user identity (e.g., user device identity) used to access multiple integrated system service providers and the individualized medicine platform module 106. As such, the access management system can be configured to execute authentication operations in a shared manner across one or more service provider applications and/or devices (e.g., using SAML/OAuth or other SSO (Single Sign-on) protocols and trust relationship mechanisms). In an aspect, individualized medicine platform module 106 can store (e.g., in one or more instance database employed across one or more server devices) profile metadata representing one or more user profile (E.g., name data, unique identifier data, address data, picture data, etc.).

In an aspect, user profile data can be stored across multiple instance databases, however, access management system can allow for a single login (e.g., authentication mechanism and provider) to be shared across different instance databases. Furthermore, in an aspect, access management system can store user credential metadata (e.g., login ID, password, digital certificates or biometrics such as fingerprints, face ID etc.) representing authentication information in one or more credential data store (only associated with access management system) of the access management system. In an aspect, the credential data store can store user credential data for all users (across all integrated systems and platforms) as well as groups of users. As such, access management system can execute authentication operations across all users (e.g., pharmaceutical client users, website users, customer support users, etc.). In a non-limiting embodiment, a user can refer to a device accessing (e.g., via login) the individualized medicine platform module 106 as a representative of an institute to execute various operations within a step of the individualized medicine supply chain for a therapy in a respective region. Furthermore, a user can be a member of a group in an identity provider (e.g., pharmaceutical company, hospital, infusion center, manufacturing facility, etc.) where such membership is mapped to a role (e.g., role 1 can access personal health identifier (PHI) information for therapy A, role 2 is restricted from accessing PHI for therapy A) within such identity provider corresponding to respective permissions (e.g., executable actions or operations permitted by user) on a therapy in a region.

In an aspect, an identity provider (IDP) can refer to an entity configured to create, maintain, and manage identity information for user profiles and devices of individualized medicine platform module 106 while providing authentication services to integrated applications (e.g., within a federated or distributed framework in various non-limiting embodiments). In an aspect, an IDP is configured to provide a verification source of user credentials for individualized medicine platform module 106 and integrated applications. As such, an IDP can provide a trust relationship (and in some instances a single-sign on protocol to facilitate execution of authentication operations for individualized medicine platform module 140-2 and any number of respective integrated components (e.g., using integration module 140-2). In an aspect, integration module 140-2 can enable an assignment of permissions to a range of users. Accordingly, a user device need only access the platform module 106 to access authentication activities for any number of platform subsystems.

For instance, a system administrator user can have escalated permissions to change administrator identities or add administrators. In another instance, integration module 140-2 can assign an identity administrator (also referred to as an on-boarder) permissions to add, change or remove other users from the platform, and assign or revoke permissions and roles to/from other users, however, the identity administrator may not remove system administrators in some embodiments. In yet another aspect, integration module 140-2 can assign an end user roles and permissions to execute day to day operational tasks via the individualized medicine platform module 106.

In yet another non-limiting embodiment, individualized medicine platform module 106 can also employ analytics module 150-2 that can acquire data (e.g., data associated with individualized medicine platform 106), curate the acquired data, generate queries for various types of analytics, determine data to include in a response to such queries, and generate output information responsive to the generated queries. In an aspect, analytics module 150-2 can employ machine-learning algorithms and/or models to extract data from curated data sets in response to multiple generated queries performed by analytics module 150-2. In an aspect, the data extracted using the queries can be utilized to identify various insights based on the application of machine-learning algorithms and/or models. In another aspect, the insights can be integrated and transformed into other insights.

Furthermore, in an aspect, the insights can be used to generate analytics output information (e.g., charts, graphs, video, audio, etc.). As an example, machine learning algorithms can be applied to historical data to perform predictive analysis of whether sufficient material supplies (e.g., AAV vectors) and other resources are available to manufacture a final product therapeutic. Furthermore, in another aspect, real world evidence data (e.g., longitudinal patient experience data, treatment data, outcome data, clinical data, genomic data, other data generated from patient experiences outside of clinical trials) can be utilized to inform learnings of machine learning algorithms (e.g., adjust model parameters or hyper parameters) to facilitate the execution of more effective predictions, define a efficacy of a model, extract learnings from data, adjusting predictive modeling problems, and other such utility. Furthermore, in another aspect, real world data can be utilized as a feedback loop mechanism to make data quality assessments, adjust analytical models or tweak the mechanism by which modules execute various operations or tasks.

In an aspect, the term module is used to denote any combination of software, hardware and/or firmware that can be configured to provide the corresponding functionality such that individualized medicine platform module 106 and client individualized medicine module 180 can be implemented using any of these combinations. In various implementations, individualized medicine platform module 106 can correspond to a client application that renders a user interface (e.g., using display module 190) on a corresponding display device of computing device 104, and communicates over a network to a server application, such as individualized medicine platform module 106. Alternatively, or additionally, client individualized medicine module 180 can represent a stand-alone application that includes the functionality of individualized medicine platform module 106 onto a same device. In one or more implementations, server(s) device 102 can represent one or more server that distribute various aspects of the individualized medicine platform module 106 across the multiple devices and/or provide cloud-based services to multiple user devices.

In another aspect, server device(s) 102 can comprise first communication module 170 configured to communicate with external devices and can represent a combination of hardware, software, and/or firmware that are configurable to facilitate the exchange of information, such as images, addresses, audio, video, commands, queries, messaging, generation of analytics and other such information. In another aspect, first communication module 170 can include one or more protocol stacks associated with a network over which data is exchanged, firmware that drives hardware to generate signals and/or process messages used in maintaining a wireless and/or wired communication session, etc. In some implementations, first communication module 170 can include computer networking ports, such as a Transmission Control Protocol (TCP) port, a User Datagram Protocol (UDP) port, a File Transfer Protocol (FTP) port, a Hypertext Transfer Protocol (HTTP) port, an Internet Message Access Protocol (IMAP) port, and so forth. Various implementations of first communication module 120-2 can include physical communication ports, such as a serial port, a parallel port, a Universal Serial Bus (USB) port, a keyboard port, a display port, an audio port, etc. In various implementations, server device(s) 102 can use first communication module 120-2 to connect with other devices over network component 114, such as computing device(s) 104.

In an aspect, network component 114 can represent any suitable type of communication network that facilitates a bi-directional link between various computing devices. Furthermore, network component 114 can include communication networks interconnected comprising elements such as a WLAN, ethernet access, wireless telecommunication network interconnected with the internet, Wi-Fi access point connected to the Internet, an IOT network, and other such networks. In another aspect, computing device(s) 104 can include client analytics module that represents user profile access to some, or all of the functionality provided by analytics module 150-2. In another aspect client analytics module can represent a browser that remotely logs onto a website hosted by server device(s) 102. Further, while client analytics module and analytics module 150-2 are illustrated as residing on separate devices, some implementations combine some or all the respective module functionality into a single computing device as further described herein.

In various implementations, computing device(s) 104 can use client analytics module to access cloud-based services provided by server device(s) 102 to obtain individualized medicine data, insights generated from the data, and other information (e.g., key performance indicators) further described herein. In another aspect, client individualized medicine module 180 can includes input module 192 to provide user access into features provided by analytics module 150-2 such as inputting a search query, inputting user feedback, requesting reports, accessing a dashboard and/or corresponding reports, scheduling data curation, scheduling data analysis, ordering data curation, ordering data analysis, transportation data curation, transportation data analysis, scoring vendor performance, adding databases for data curation, and so forth. In another aspect, computing device(s) 104 can include second communication module 196 to facilitate communications over network component 114. As one, example, computing device 104 can use second communication module 196 go communicate with individualized medicine platform module 106. Furthermore, in an aspect, second communication module 196 can represent any suitable combination of hardware, software, and/or firmware that is configurable to facilitate data exchanges with other devices.

In a non-limiting embodiment, individualized medicine platform module 106 can comprise supply chain optimization module 110-2, commercial scale module 120-2, custody & identification module 130-2, system integrations module 140-2, and analytics module 150-2 to work in concert to provide medicine supply chain monitoring, tracking, scheduling, ordering, analytics, querying, and other interactive features. Some combinations of these modules communicate with one another to exchange information, such as by defining data structures according to a set of rules to provide a mechanism for cross-entity data sharing, as well as predictable and repeatable processing by different entities to achieve expected results. For example, the set of rules can outline the type of information the supply chain data included in the data structure describes, an amount of supply chain data stored within the data structure, a format in which the supply chain data is stored within the data structure, and other such information types.

By following these rules, a first entity can create and store a data structure such that a second entity can successfully access and interpret the data included in the data structure. A data structure can include any suitable type of structure in which data can be stored, defined, and/or retrieved, such as an object, a vector, a table, a tree, a graph, a queue, a linked-list, a record, a union, a set, a string, a list, an array, a container, a matrix, a function parameter, a heap, and other such structures. In an aspect, server device 102 can include an ordering module 110-2 that acquires, processes and curate's data associated with ordering activities in a personalized medicine supply chain. For example, the supply chain can include the acquisition and generation of order data associated with ordering a courier to pick up or drop off a collection sample. In another aspect, server device 102 can include an ordering module 110-2 that acquires, process and curate's data associated with ordering activities in a personalized medicine supply chain. For example, the supply chain can include the acquisition and generation of order data associated with ordering a courier to pick up or drop off a collection sample.

In an aspect, a patient's blood sample with cells may be collected at a center of excellence (e.g., hospital) and transported to a manufacturing facility to manufacture the personalized therapeutic medicine and the personalized therapeutic medicine must again be transported back to a COE for infusion into a patient. In an aspect, supply chain optimization module 110-2 can acquire order data representing a wide range of ordering information such as shipment destination, shipment origin, storage conditions during transport, required temperature conditions during transport, handling instructions, instructions to place an order with a particular carrier, type of transportation mode (e.g., ground transport, air transport, etc.), sample information (e.g., quantity, type of sample, etc.).

Furthermore, in an aspect, custody & identification module 130-2 can acquire, generate, curate, store, and query chain of identity information (e.g., patient identification information corresponding to a sample, patient identification information corresponding to a personalized medicine, medicine specifications, courier identification information, manufacturer identification information, storage facility identification information, supplier identification information of raw material, etc.), chain of custody information (e.g., temperature data, tilt data of package, tracking a sample or medicine custodian at a given point in supply chain, etc.), and other such ordering information. In an aspect, this non-limiting example of an ordering feature of individualized medicine platform module 106 illustrates how any suitable type of order data can be acquired, generated and/or stored by ordering module 110-2 and how modules can perform operations in combination with other modules. In an aspect, server(s) device 102 can include database device(s) 160 to represent any suitable source of data and/or information. Alternatively, or additionally, database device(s) 160 can represent storage for data generated by the medicine supply chain module 106.

Furthermore, also disclosed herein is a smart label device(s) 198. In an aspect, smart label device(s) 198 can represent a device comprising a digital display capable of affixing or adhering to a package, material, specimen, sample or item (referred to as any of the foregoing throughout the disclosure). The smart label device(s) 198 can be configured to display update data (e.g., de-identified information, PHI, or other such information) or content at the display based on label requirement rule sets. As such, smart label device(s) 198 can display different content at different facilities, to different users or custodians of an item and smart label device(s) 198, or locations to which the smart label devices are located.

In a non-limiting embodiment, smart label device(s) 198 can be configured with an electronic ink (e-ink) programmable display having low power requirements to display content. Furthermore, smart label device(s) 198 can be powered by a range of battery technologies that allow power to transmit to the smart label device(s) 198 for several weeks or longer. In another non-limiting embodiment, smart label device(s) 198 can include a GSM/3G radio antenna with added remote commands. Furthermore, smart label device(s) 198 can utilize a tamper-resistant hardware to prevent tampering with the label or label information. In another non-limiting embodiment, the smart label device(s) 198 can include hardware-based cryptographic tokens to perform two-factor authentication of one or more user of smart label device(s) 198.

For instance, a user device requesting access to updated label information can transmit input data representing a personal identification number (PIN) displayed on a token identification device, to a smart label module 108 of server device(s) 102. The smart label module 108 can perform validation operations that approve of the input PIN as valid or disprove of the number as invalid. Upon validation of the PIN, the smart label module 108 can transmit updated data to smart label device 198 for rendering of such updated label information at a display (e.g., E-ink display). In other non-limiting embodiments, other authentication forms including multi-factor authentication tools can be utilized by smart label device(s) 198 to protect unauthorized information from being displayed at unauthorized times and by unauthorized viewers.

In another aspect, smart label device(s) 198 can employ any of a range of display types including an organic thin-film transistor (OTFT), E-ink display with flash memory storage, E-ink panel capable of representing images and/or two-dimensional codes, bi-stable LED display, LCD display shutter or LCD screen, TFT display for freezer use, displays that employ a RGB color model, and other such displays. In another aspect, the display of smart label device(s) 198 can render a range of content or updated content such as facility data (e.g., identification of facility or user such as cryogenic storage facility, manufacturing facility, collection center, apheresis center, raw material facility, infusion site, hospital, clinic, etc.), destination data (e.g., a name of a person or organization intended to receive the item), first address data (e.g., street address), second address data (e.g., city, state, zip code information), retrieval data (e.g., date for item pickup), courier data (e.g., identity of courier organization or personnel to pick-up item for transport, mode of transport, etc.), status data (e.g., package or item status or standing at a particular point in time), descriptive data (e.g., identifier of the item and/or title of the product as well as alphanumeric identifier), bar code data (e.g., visual identification symbol configured to be read by a scanner, other line of sign technologies, etc.), and/or quick response (QR) code data (e.g., code configured to store data such as character data).

In another aspect, smart label device(s) 198 can comprise any one or more sensor technology such as generic sensors, temperature sensors (e.g., temperature tracking of items during transportation), humidity sensors (e.g., monitoring preservation conditions of a sample), light intensity sensors (e.g., monitoring of light intensity exposure of the item), passive light sensors, accelerometers (e.g., latching bi-stable accelerometer, etc.), speed sensors (e.g., to detect a vehicle speed), color sensor (e.g., semiconductor based), photodiodes, ultrasonic beacons, and other such sensors. In another aspect, smart label device 198 can employ a range of sensors to detect environmental condition data (e.g., temperature, humidity, noise, etc.).

Furthermore, smart label device(s) 198 can employ activation sensors and/or actuator sensors such as movement sensors (e.g., gyroscopes, accelerometers, etc.) or pressure sensors to detect pressure or movement information associated with the item. In an aspect, smart label device(s) 198 can facilitate the detection of conditions pursuant to a set of condition rules corresponding to the contents of a package (e.g., biological materials needing to be stored at temperatures within a target temperature range, delicate specimens that must not be exposed to turbulence or target altitudes, etc.). In an aspect, smart label device(s) 198, can communicate sensor data to smart label module 108. Furthermore, smart label module 108 can determine whether the package contents are subjected to favorable or unfavorable conditions. For instance, smart label module 108 can compare temperature data, speed data, motion data, light intensity data, or vibration data to threshold data values for each respective parameter or a threshold value for a combination of parameters to determine the threat of various conditions (as detected by the sensors) to the quality of the package contents (e.g., biological material). Furthermore, smart module 108 can trigger a notification to various stakeholders based on detected sensor data.

As an example, if a courier of such package is an autonomous vehicle, the courier can trigger a deceleration in speed or determination of an alternative path to maintain a slower or steadier ride to preserve the condition of the package contents. In other non-limiting embodiments, smart label module 108 can control other devices based on detected sensor information from smart label device(s) 198. For instance, smart label module 108 can trigger adjustments to control the temperature of cryogenic storage compartments or refrigerated compartments based on a detected condition of the package contents as detected by smart label device(s) 198. In another non-limiting embodiment, smart label module 108 can control manufacturing processes associated with generating a personalized therapy based on detected conditions of package contents (e.g., half-life of biological materials), such controls include adjusting a speed of assembly, adjusting an order of manufacturing operations, suggesting additional steps to improve a quality of the therapeutic (e.g., centrifuging the material). In an aspect, smart module 108 can control such additional devices via communicative coupling to such other devices (e.g., manufacturing equipment, cryogenic storage equipment, etc.).

In a non-limiting embodiment, smart device(s) 198 can continuously detect data from respective smart label sensors. Furthermore, smart module 108 can continuously determine package content (e.g., package can refer to a box, blood bag, test tube, vile, or other containment modality used to store biological material) conditions based on an analysis of the continuous detected data (e.g., threshold comparisons and evaluation). The smart label module 108, can control and/or configure smart label device(s) 198 and other equipment (e.g., manufacturing equipment, cryogenic storage equipment) to execute an operation (e.g., increase or decrease a temperature or speed of manufacture, etc.) based on the detected data exceeding or registering below a target range. Furthermore, smart module 108 can detect geo-location coordinates to automatically trigger an adjustment of various conditions based on predicted parameter corresponding to such geo-location coordinates. Furthermore, smart module 108 can also access smart label information to display at a display interface of smart label device(s) 198 based on a prediction of geo-location coordinates to be reached at a target time. As such, smart module 108 in connection with smart label device(s) 198 can avert technical problems associated with the deterioration of quality of patient materials due to transportation, storage, and/or manufacturing processes.

Furthermore, in an aspect, smart label device(s) 198 and corresponding sensors can be utilized in a range of cases. For instance, smart label device(s) 198 sensors can be utilized in individualized medicine supply chains (e.g., tracking patient materials and packages), retail uses (e.g., labels to display and indicate information to consumers such as price fluctuations), logistics (e.g., product management tasks that receive data from the way a consumer interacts with the product and corresponding label), provide instructions related to the product to which it is affixed, perform item identification operations, and other such uses. In some non-limiting embodiments, smart label device(s) 198 can be configured with hardware that can perform smart label functions at extreme cold temperatures to withstand cryogenic preservation conditions (e.g., temperatures as low as −190 degrees Celsius). Furthermore, temperature sensors of smart label device(s) 198 can allow them to detect storage temperatures and smart label module 108 can compare such temperature data to threshold temperature data to determine whether the condition the material corresponding to smart label device(s) 198 is satisfactory or in jeopardy.

In one or more non-limiting embodiments, smart label device(s) 198 can also include one or more controller components such as a complementary metal-oxide-semiconductor (CMOS), hybrid CMOS, microcontroller, low frequency CMOS controller, e-paper driver, microcontroller, and/or other such controller component. In yet another aspect, smart label device(s) 198 can employ any of a range of communication technologies to perform communication operations with other devices (e.g., server device(s) 102, computer device(s) 104, individualized medicine platform module 106, smart label module 108, etc.). For instance, smart label device(s) 198 can comprise any of the following technological components or utilize any of the following technologies: radio-frequency identification (RFID), Ai-Net wireless protocols, Wi-Fi, Radio Force X4 (RFX4), infrared, nearfield communication (NFC), ultra-high frequency (UHF) RFID, and other communication modalities. In other non-limiting embodiments, smart label device(s) 198 can comprise any one or more of a range of battery technologies or power saving technologies including but not limited to lithium batteries, rechargeable batteries, duty-cycling power technologies, solar cell technologies, and photovoltaic cell technologies. As such, smart label device(s) 198 can include a range of structural elements and can be configured to operate in connection with a smart label module 108. In some non-limiting embodiments individualized medicine platform module 106 can employ smart label module 108. In other non-limiting embodiments, smart label module 108 can be a platform application executing on a server and communicatively couple with individualized medicine platform module 106.

In another implementation, computing device(s) 104 can include client individualized medicine module 180 and second communication module 196. In an aspect, client individualized medicine module 180 can employ display module 190, input module 192, and/or client smart label module 194. The non-limiting example environment 100A can include an example system that can be used to configure, transmit data to/from, and/or control smart label device(s) 198. In other non-limiting embodiments, server device(s) 102 can form part of an interconnection architecture such that seamless operations, and configurations can be implemented to multiple smart label device(s) 198. Furthermore, users across multiple devices can access the functionality enabled by client smart label module 194 and corresponding functionality executed by smart label module 108. For instance, thousands of users can access client smart label module 194 to request smart label module 108 (executing on server device(s) 102) to query transportation data acquired from thousands of corresponding smart label device(s) 198 in a seamless and efficient manner.

In other non-limiting embodiments, environment 100A can comprise distributed scheduling system 103 executed in connection with the individualized medicine platform module 106. In an aspect, distributed scheduling system 103 can employ one or more engine module to facilitate execution of distributed scheduling activities, schedule predicting and time duration predicting for various scheduled activities, and re-scheduled activities in connection with an individualized medicine supply chain. Distributed scheduling system 103 includes distributed scheduling engine module 110-1, rescheduling engine module 120-1, capacity engine module 130-1, load balancing engine module 110-1, and prediction engine module 150-1 that work in concert to provide distributed scheduling operations in accordance with one or more implementations. Some combinations of these modules communicate with one another to exchange information, such as by defining data structures according to a set of rules to provide a mechanism for cross-entity data sharing, as well as predictable and continuous scheduling across different entities, to achieve expected results.

The term “module” refers to any combination of hardware, software, and/or firmware that can be configured to provide the corresponding functionality such that the distributed scheduling system 103 and/or client distributed scheduling module 195 can be implemented using these hardware, software, and/or firmware combinations and such that distributed scheduling system 103 and respective modules can be configured, controlled and/or operated using such combinations as well.

For example, distributed scheduling engine module 110-1 can execute a set of rules that determine which entity can generate and store a data structure such that other entities can successfully access and interpret the data included in such data structure. Furthermore, distributed scheduling engine module 110-1 can provide for data structures that limit the amount of data stored within such structure and the format in which such data must take. In an aspect, a data structure can include any suitable type of structure in which data can be stored (e.g., array, string, file, bitmap, object, etc.). Accordingly, distributed scheduling engine module 110-1 can be configured as an abstraction layer that aggregates, curates, and analyzes capacity data and scheduling data sourced from a distributed array of databases and/or data stores. For instance, distributed scheduling engine module 110-1 can access and source scheduling and capacity data from one or more patient databases, collection site and/or infusion site databases 122, courier databases 124, and manufacturing site databases 126.

Furthermore, distributed scheduling engine module 110-1 can use such structured information to execute optimal scheduling reservations that enhance operability of an individualized medicine supply chain associated with generating an individualized therapy. In another aspect, distributed scheduling engine module 110-1 can execute determinative thresholds to determine whether to aggregate and analyze data at runtime (i.e. on-demand only when needed) or perform such operations a-priori. For instance, distributed scheduling engine module 110-1 can aggregate scheduling and capacity data in accordance with an a-priori model that identifies patterns and associates such patterns with asset of data sourced from respective distributed sources. Furthermore, distributed scheduling engine module 110-1 executing the a-priori model can understand the most frequent data sets that are sourced and source such data sets associated with those frequent data sets. For instance, if a patient name and date of infusion is sources, then the distributed scheduling engine module 110-1 employing the a-priori model can also source the courier schedule data associated with scheduled delivery of blood for infusion. As such a frequency model that source all items from distributed sources together can allow for faster processing and data access operations.

In another aspect, an analysis of capacity data and scheduling data by distributed scheduling engine module 110-1 at run-time can avoid aggregating millions of data sets in a single instance which enhances performance of processing and memory access operations of the system. Furthermore, this allows for retrieval of information from an organized and combined database at the time an analysis is needed which saves computation expense vs retrieving from an unorganized source, reduces sizes of source databases, provides a significantly better performance and resilience than a source database and can be resource efficient where a determined quantity of resource needs to run such aggregation and analysis process.

The more resources needed, the greater the time sensitivity of the aggregation is better determined (e.g., spaced out) to prevent slowing down aggregation processes and facilitate expedited scheduling operations. In another aspect, distributed scheduling engine module 110-1 can facilitate a determination of scheduling availability based on available capacity information and patient schedule to identify and reserve scheduling slots in distributed systems. Furthermore, a booking record can be created in the distributed scheduling engine module 110-1 with references to the bookings in the distributed systems.

In another implementation, distributed scheduling engine module 110-1 can apply machine learning algorithms, data mining algorithms and Principal Component Analysis (PCA) algorithms to identify data relationships between acquired scheduling data from distributed sources. Furthermore, distributed scheduling engine module 110-1 can utilize machine learning algorithms to label sets of scheduling data, compare set of scheduling data for similarities, group sets of data based on similarities, and perform other such activities. In another aspect, distributed scheduling engine module 110-1 can acquire relevant data from a distributed array of sources based on efficiency and reliability indicators of data transmission. For instance, distributed scheduling engine module 110-1 can consider connectivity factors such as distributed sources latency (e.g., low latency is prioritized) and service reliability scores of carriers. Furthermore, distributed scheduling engine module 110-1 can employ mapping rules to ensure sourcing of scheduling data can be prioritized and queued via a handler that optimizes the sourcing based on a priority scheme where fastest resource sources have higher sourcing priorities than lower resource sources.

In another implementation, distributed scheduling system 103 can implement rescheduling engine module 120-1. As such, such module can execute rescheduling operations and determine an impact of rescheduling a scheduled operation. In an aspect, rescheduling engine module 120-1 can utilize predictive data representing a likelihood of a patient, courier, or other stakeholder along the personalized medicine supply chain to reschedule in determining to execute a rescheduling operation. Furthermore, rescheduling engine module 120-1 can base rescheduling determinations on capacity data or resource availability data. For instance, rescheduling engine module 120-1 may impose rules that require qualified health care professional to be available at the aphaeresis site and a particular provider to be available at the infusion site before rescheduling a blood draw appointment for a patient.

Furthermore, rescheduling engine module 120-1 may utilize a determination of manufacturing capacity or availability of raw materials to perform a manufacturing activity related to generating a personalized medicine therapeutic for a patient before executing a rescheduling operation for blood draws, which, such blood will ultimately need to be scheduled for use to manufacture into a therapeutic product within a predefined time window. As such, in various implementations distributed scheduling engine module 110-1 can employ iterative processes to curate and analyze metadata, data models, and relational data associated with distributed scheduling and capacity databases. Furthermore, rescheduling engine module 120-1 can utilize such curated capacity data, scheduling data, relational data, predicted rescheduling likelihood data and other relevant data to execute rescheduling of various operations (e.g., patient blood or sample draws, courier pick-up, courier drop-off, cryogenic freezing of sample, transportation of raw materials, manufacturing of therapeutic, infusion of therapeutic, etc.) throughout the individualized medicine supply chain.

In yet another implementation, distributed scheduling system 103 can employ a capacity engine module 130-1 configured to access, retrieve and analyze capacity data from disparate systems at runtime. Furthermore, capacity engine module 130-1 can employ various techniques to enhance processor speeds and capacity data access speeds by caching predefined capacity data types at various time intervals and enabling the publication of capacity data to capacity registries. Accordingly, capacity engine module 130-1 can curate capacity data and distributed scheduling engine module 140-1 can access such data to execute scheduling activities. In another implementation, distributed scheduling system 103 can employ load balancing engine module 140-1 configured to execute load balancing operations across various manufacturing facilities.

For instance, load balancing engine module 140-1 can determine whether there is adequate production capacity available across several distributed manufacturing facilities to meet scheduled manufacturing demands. Furthermore, load balancing engine module 140-1 can determine whether components and sub-assemblies are in stock, available, need be acquired and determine or assume lead times required to provide such components for manufacturing. As such, load balancing engine module 140-1 can execute load balancing operations to balance throughput rates of activities within an individualized medicine therapeutic generation process. Accordingly, load balancing engine module 140-1 can determine and balance loads associated with courier activities (e.g., transportation and shipping, etc.) across multiple distributed courier systems and manufacturing activities across multiple distributed courier systems. Furthermore, distributed scheduling engine module 140-1, rescheduling engine module 120-1, and capacity engine module 130-1 can execute scheduling, rescheduling and capacity curating operations based on load balancing engine module 140-1 determinations and considerations.

In another implementation, distributed scheduling system 103 can employ prediction engine module 150-1 to execute one or more prediction model to estimate a likelihood of rescheduling events to occur in relation to patient activities (e.g., blood draws, etc.), time duration for various activities (e.g., material collection, material transportation, other transportation lags, etc.), manufacturing activities (e.g., manufacturing system integration, final product assembly, etc.), location prediction of items (e.g., location of materials, samples, products, location within manufacturing process, etc.), prediction of time duration to batch multiple orders, and other such predictions. Furthermore, prediction engine module 150-1 can predict delays across the individualized medicine therapeutic supply chain to optimize capacity (e.g., using capacity engine module 130-1) and scheduling operations (e.g., using distributed scheduling engine module 140-1).

Turning now to FIG. 1B, illustrated is an example environment 100B in accordance with one or more implementations. In various implementations, the example described with respect to FIG. 1B can be considered a continuation of the example described in FIG. 1A. Repetitive description of like elements employed in other embodiments described herein is omitted for sake of brevity. FIG. 1B illustrates an example, non-limiting block diagram of a representative environment 100B in which a distributed scheduling system 103 associated with an individualized medicine platform module 106 and a blockchain system 132 can be utilized in accordance with one or more implementations described herein.

In an aspect, environment 100B can include or otherwise be associated with one or more server device(s) 102 that can execute individualized medicine platform module 106, smart label module 108, distributed scheduling system 103 and associated modules such as supply chain optimization module 110-2, commercial scale module 120-2, custody & identification module 130-2, system integrations module 140-2, analytics module 150-2, distributed scheduling engine module 110-1, rescheduling engine module 120-1, capacity engine module 130-1, load balancing engine module 140-1, prediction engine module 150-1 and first communication module 170. Furthermore, server device(s) 102 can comprise database(s) 163. Environment 100B can also include computing device(s) 104 that employs client individualized medicine module, client smart label module 194, client distributed scheduling module 195, input module 192, display module 190, and second communication module 196. Environment 100B can also include network component 114, smart label device(s) 198, collection site and/or infusion site databases 122, courier databases 124, and manufacturing site databases 126. Furthermore, in an aspect, Environment 100B can include blockchain 132 and blockchain node(s) 134.

In an aspect, blockchain 132 can be generated in accordance with one or more implementation disclosed herein. Furthermore, blockchain 132 can comprise blockchain node(s) 134. In a non-limiting embodiment, environment 100B can be configured to communicate and/or integrate with a distributed blockchain system comprising multiple computing nodes, such that each computing node is configured to store a copy or a portion of a blockchain. In some instances, a blockchain 132 may be used to store data associated with distributed scheduling system 103, collection site and/or infusion site databases 122, courier databases 124, manufacturing site databases 126, smart label device 198, individualized medicine platform module 106, smart label module 108 and other such devices in environment 100B. Furthermore, in an aspect, blockchain 132 can be a distributed database that maintains a continuously updated list of data records (e.g., data of manufacturing site/infusion site/collection site/center of excellence site capacities and schedules, scheduling reservations, pending reservations, predictive data, time estimates, list of smart label device locations, smart label device sensor data, smart label device data user authentication data, geo-fencing location data, specimen condition data, cryogenic storage temperature data, courier data, etc.) where each the updated list of data records can be stored on blocks linked together as a chain. In an aspect, the blocks can be secure storage vehicles (e.g., encryption based on public-key and private-key pairings) and are immutable (cannot be changed).

In another aspect, the blockchain 132 can record a transaction (e.g., a transfer of information onto, from or with individualized medicine platform module 106, distributed scheduling system 108, smart label module 108, smart label device(s) 198) within a ledger database of the blockchain 132 that is shared by devices participating in a distributed network of computers. In yet another aspect, the distributed ledger can present a consensual (by all computers within the distributed network) record corresponding to a cryptographic audit trail that is validated and maintained by independent computers. In yet another aspect, blockchain 132 can employ a network of blockchain nodes 134 that can support the blockchain, which may include a set of blocks that store data corresponding to individualized medicine platform module 106 and distributed scheduling system 103.

In an aspect, data stored on nodes of blockchain 132 can include transactional data (e.g., scheduling data, capacity data, time duration data, scheduling data, predictive data, event data, identifier data, smart label device(s) 198 data, location data, custody data, triggering event data, etc.) acquired, generated, curated, transformed, and/or received by individualized medicine platform module 106. In a non-limiting aspect, the data can be transmitted as duplicate data to a blockchain system employing blockchain 132. In another non-limiting aspect, the data can be transmitted to the blockchain system as original data (non-duplicate data), such that the blockchain system serves as a primary data store for one or more sets of data. In an embodiment, only the additional data in a transaction (not including the previously transmitted duplicate data) can be transmitted for incorporation onto the blockchain.

As such, the blockchain can be configured to store data corresponding to the data of individualized medicine platform module 106, distributed scheduling system 103, smart label module 108, and/or smart label device(s) 198. In an aspect, blockchain 132 is a data structure that stores a list of transactions. In an aspect, blockchain 132 can be a distributed electronic ledger that records transactions between various stakeholders (e.g., providers, patients, centers of excellence, couriers or third-party logistics providers, manufacturers, suppliers, cryogenic storage facilities, administrative managers, and other such stakeholders) of the individualized medicine platform module 106. In one or more non-limiting embodiments, blockchain 132 can be a decentralized public (or private) transaction ledger that can be deployed over one or more node (e.g., server) and configured to perform transaction-based state transitions and smart contract functionality.

In a non-limiting implementation, computing device(s) 104 can interact with a server device(s) 102 configured to control one or more node (e.g., blockchain node 134 and other such nodes) of blockchain 132. In an aspect, server device(s) 102 can be configured to facilitate access to one or more node of blockchain 132. For instance, server device(s) 102 can control access to blockchain node 134 that represents an Ethereum blockchain or other distributed ledger node featuring transaction-based state transitions and/or smart contract functionality. In another aspect, a smart contract may be stored as a block with blockchain 132 and data included as components of the smart contract can be stored within separate blocks within the blockchain. Furthermore, in a non-limiting embodiment, each node of a blockchain 320 can store a copy of a smart contract program structure. In an aspect, environment 100B allows for a communication between any individual device(s) in environment 100C and blockchain 132. In other non-limiting embodiments, blockchain communication routes can be limited between select devices.

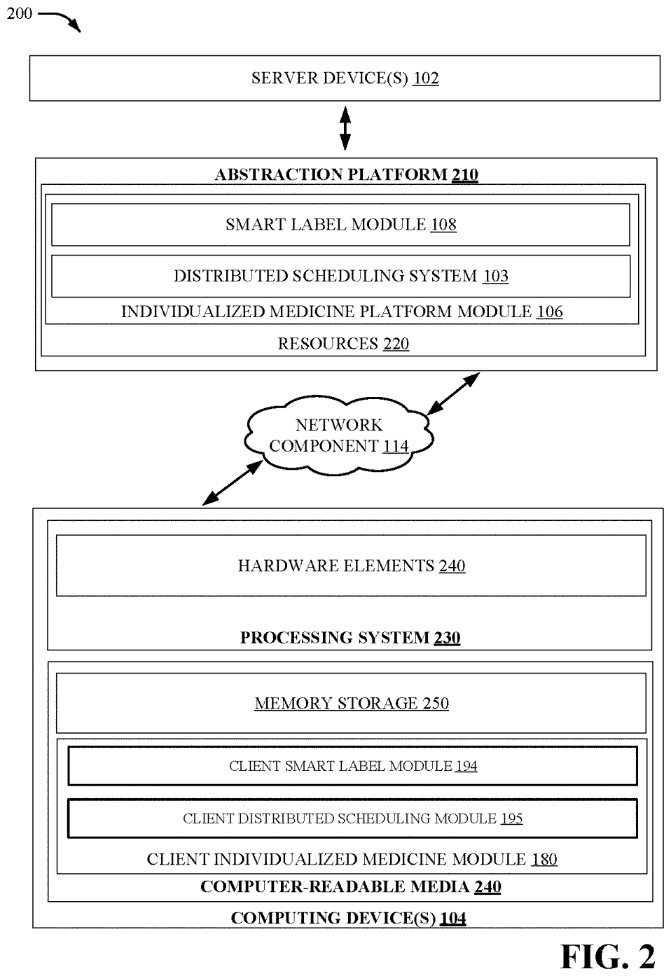

Turning now to FIG. 2, illustrated is an example environment 200 in accordance with one or more implementations. In various implementations, the example described with respect to FIG. 2 can be considered a continuation of the examples described in FIG. 1A-1B. At FIG. 2, illustrated is an example, non-limiting diagram of a representative environment 200 in which cloud-based services can be used to provide features corresponding to the automated generation of scheduling and capacity optimization operations associated with a distributed scheduling system in accordance with one or more implementations described herein. Repetitive description of like elements employed in other embodiments described herein is omitted for sake of brevity.

FIG. 2 illustrates an example, non-limiting environment 200 in which cloud-based services can be used to provide features corresponding to events and activities associated with an individualized medicine platform module 106, distributed scheduling system 103, and smart label module 108 platform in accordance with one or more embodiments described herein. In an aspect, environment 200A can include server device(s) 102, computing device(s) 104, and network component 114, where computing device(s) 104 includes a processing system 230, and one or more computer-readable media 240.

In an aspect, processor 230 can comprise one or more processor configured to perform one or more operations (of at least one module of client individualized medicine module 180) using hardware. As such, processor 230 can include hardware elements 240 that may be configured as processors, functional blocks, and so forth. This may include implementation in hardware as an application specific integrated circuit or other logic device formed using one or more semiconductors. In an aspect, the hardware elements 240 are not limited by the materials from which they are formed, or the processing mechanisms employed by such materials. For example, processors may be comprised of semiconductor(s) and/or transistors (e.g., electronic integrated circuits). In such a context, processor-executable instructions may be electronically executable instructions.

The computer-readable media 240 is illustrated as including memory storage 250. The memory storage 250 represents memory storage capacity associated with one or more computer-readable media. The memory storage 250 may include volatile media (such as random-access memory (RAM)) and/or nonvolatile media (such as read only memory (ROM), Flash memory, optical disks, magnetic disks, and so forth). The memory storage 250 may include fixed media (e.g., RAM, ROM, a fixed hard drive, and so on) as well as removable media (e.g., Flash memory, a removable hard drive, an optical disc, and so forth). The computer-readable media 240 may be configured in a variety of other ways as further described below. In an aspect, client individualized medicine module 180 of FIG. 1 is illustrated as residing within memory storage 250, client smart label module 194 and client distributed scheduling module 195, but alternate or additional implementations can implement client individualized medicine module 180 using combinations of firmware, hardware, and/or software without departing from the scope of the claimed subject matter, such as hardware elements 240.

Example environment 200 can enable multiple devices to be interconnected through server device(s) 102, where server device(s) 102 can be local to the multiple devices, remote from the multiple devices, or any combination thereof. In one or more implementations, server device(s) 102 can be configured as a cloud of one or more server computers that are connected to the multiple devices through a network (e.g., using network component 114), the Internet, or other data communication link capable of enabling functionality to be delivered across multiple devices (e.g., several smartphone devices, desktops, tablets, etc.) to provide a common and seamless experience to a user of the multiple devices. Each of the multiple devices may have different physical requirements and capabilities, and the central computing device uses a platform to enable the delivery of an experience to the device that is both tailored to the device and yet common to all devices. In a non-limiting embodiment, a class of target devices having unique physical features, types of usage or other such characteristics can be deployed, and tailored user experiences can be implemented on such class of generic class of devices.

In an aspect, cloud computing network or network component 114 can include or represent an abstraction platform 210 for resources 220. In another aspect, the abstraction platform 212 abstracts underlying functionality of hardware (e.g., servers) and software resources of the cloud 210. In an aspect, resources 220 may include applications and/or data that can be utilized while computer processing is executed on servers (e.g., server device(s) 102) that are remote from the computing device(s) 104. For example, resources 220 can include individualized medicine platform module 106, smart label module 108, and distributed scheduling system 103 at FIG. 1A. In another aspect, the abstraction platform 210 may abstract resources and functions to connect computing device 104 with other computing devices and may also serve to abstract scaling of resources to provide a corresponding level of scale to encountered demand for the resources 220 that are implemented via the abstraction platform 210. Accordingly, in an interconnected device embodiment, implementation of functionality described herein may be distributed throughout the system. For example, the functionality may be implemented in part on the computing device 104 as well as via the abstraction platform 210 that abstracts the functionality of the network component 114.

In another aspect, abstraction platform 212 can allow for external system integrators to keep data private on individualized medicine platform module 106, distributed scheduling system 103, smart label module 108 while abstraction platform 210 can extract learnings from such data to educate a range of machine learning algorithms. For instance, hyper-parameters of machine learning models applied to a first external device data can be extracted and the hyper-parameters can be applied to data of a second external device. As such, learnings from analyzing and curating a first client data can be applied to the analysis and curation of a second client data without ever exposing the private data of each user.

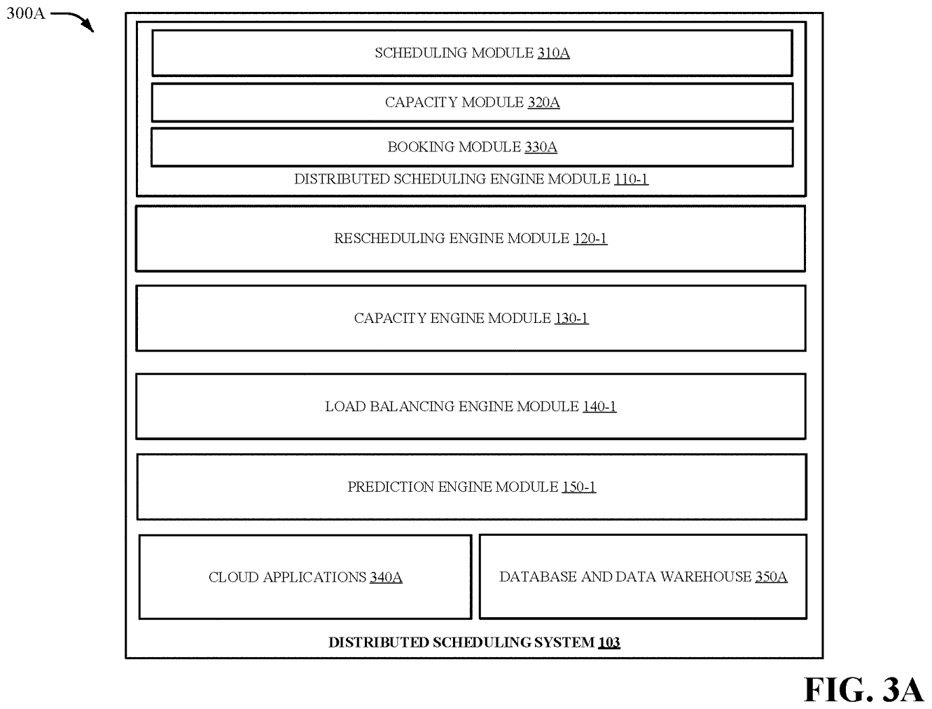

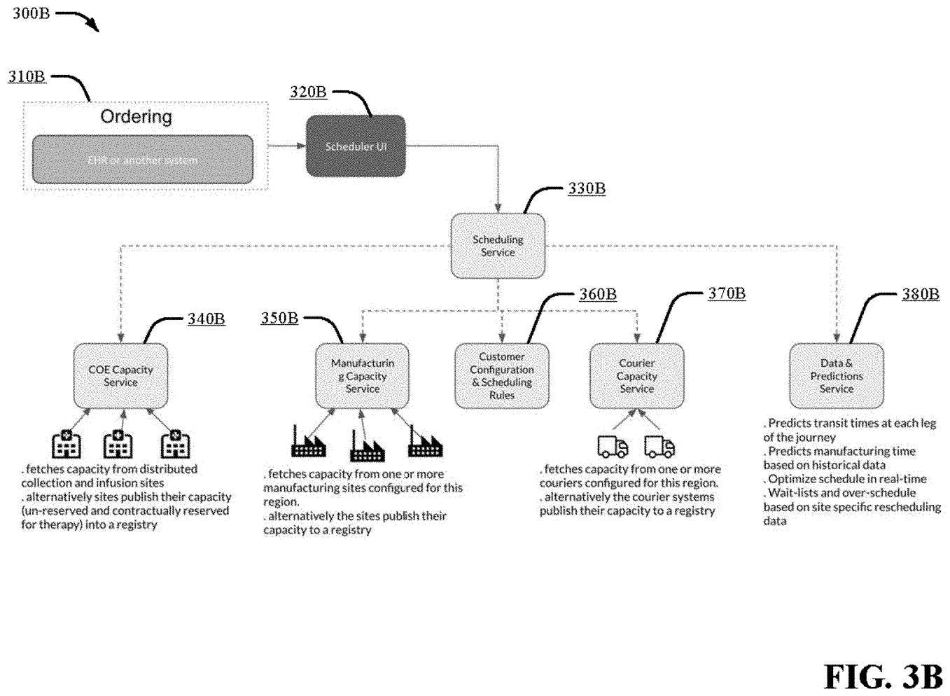

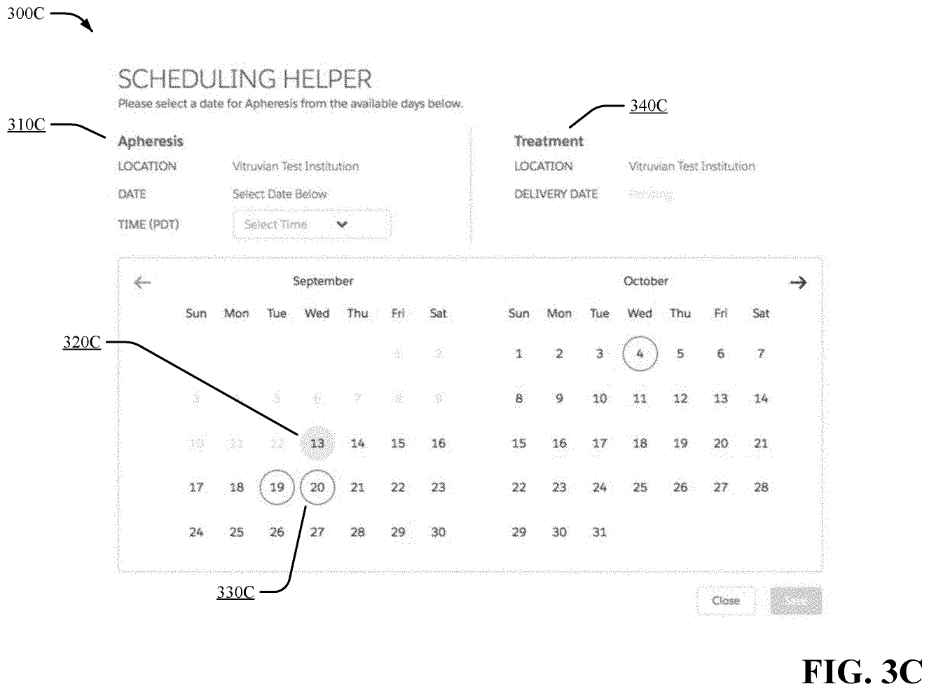

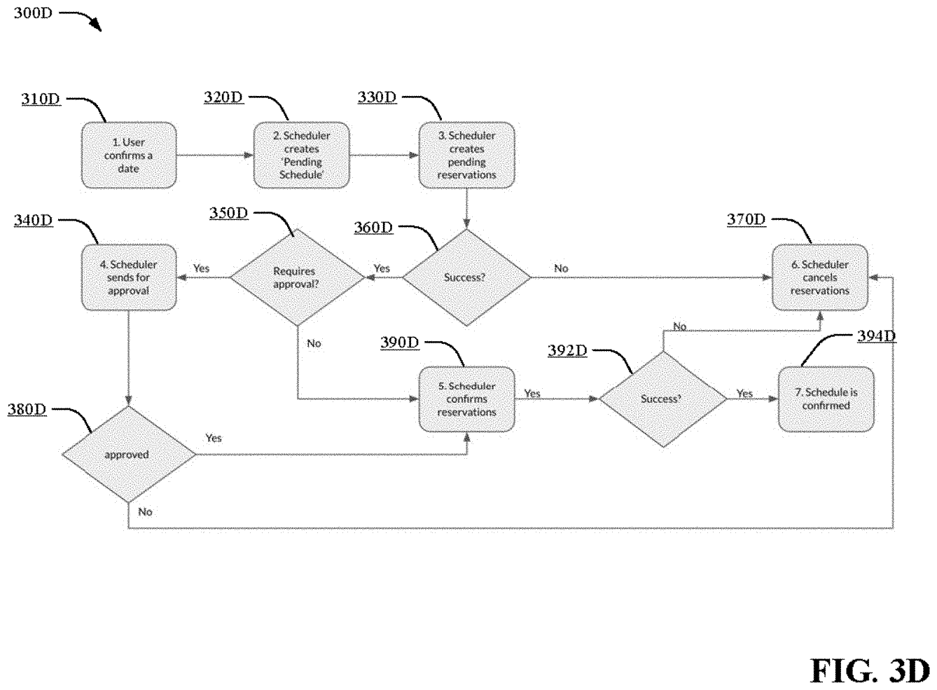

FIG. 3A illustrates an example, non-limiting example block diagram of a distributed scheduling system 300A and an example distributed scheduling engine module in accordance with one or more implementations described herein. In various scenarios, the example described with respect to FIG. 3A can be considered a continuation of one or more examples described with respect to FIGS. 1A-2. Repetitive description of like elements employed in other embodiments described herein is omitted for sake of brevity.

FIG. 3A includes a distributed scheduling system 103 that can be implemented using various combinations of computing devices and/or modules, such as varying combinations of servers 102, computing device(s) 104 of FIG. 1A. The distributed scheduling system 103 includes distributed scheduling engine module 110-1, rescheduling engine module 120-1, capacity engine module 130-1, load balancing module 140-1, prediction engine module 150-1, cloud applications 340A, database and data warehouse 350A. In an aspect, distributed scheduling engine module 110-1 can generally scan data sources, such as database and data warehouse 350A and/or cloud applications 340A, to identify source data that is subsequently curated, acted upon, and/or enriched by other modules such as prediction engine module 150-1. For instance, distributed scheduling engine module 110-1 can employ scheduling module 310A to access and source scheduling data from disparate systems at runtime. Furthermore, capacity module 320A can access and source scheduling data from disparate systems at runtime. For instance, capacity module 320A can access, based on a query or occurrence of a triggering event, manufacturing capacity data that represents the availability of respective therapeutic manufacturing process at a manufacturing site during a given time slot.

Furthermore, distributed scheduling engine module 110-1 can employ such scheduling module 310A and capacity module 320A to cache capacity data on a recurring basis based on a predetermined time interval and refresh the software and/or hardware cache of a storage device based on the time interval occurring or upon occurrence of a prompt by a host system that supports a scheduling database or capacity database. In an aspect, the capacity data cache can be used to temporarily select data for fast access. As such, capacity module 320A can curate capacity data from several distributed sources at a capacity registry. Furthermore, several distributed manufacturing sites and provider databases can publish capacity data to such registry. In another aspect, the capacity registry can be configured to isolate private data within the database based on privacy factors (e.g., client therapy manufacturer identity) to satisfy security and privacy requirements.

In another aspect, scheduling engine module 110-1 may source scheduling and capacity data from various sources at different speeds due to a range of factors related to the source systems. In some implementations, for those sources that are slow or in other implementations for all sources of scheduling data and capacity data, capacity module 32A can source data a capacity registry such that caching service time can be reduced. As an example, capacity module 320A can cache capacity data based on the type of storage media used for the cache, cache location within a storage media, data storage density, as well as read and write speed that can be realized in transferring data to and from the cache. As such, scheduling engine module 110-1 can reduce data access times to perform scheduling operations by accessing some or all capacity data from the cache registry and other data directly from the data source (e.g., database, data store, third party system caches, etc.). Given the volume of data analyzed and accessed by scheduling engine module 110-1, the cache registry can be used to read and write large amounts of data at faster speeds.

In another aspect, capacity module 320A can generate a capacity registry for distributed capacity providers in various respects of an individualized medicine supply chain. For instance, capacity module 320A can generate and source a center of excellence capacity service registry, manufacturing capacity service registry, courier capacity service registry, and data and predictions. As an example, a center of excellence capacity registry can allow for the sourcing, publication, aggregation and curation of data associated with an array of collection site databases and infusion site databases. Similarly, a manufacturing capacity service registry can be utilized by manufacturing sites for publication of its data. In some implementations, scheduling module 310A and capacity module 320A can employ a blockchain network to capture capacity registry data and other such registry data (e.g., from various industries). Furthermore, the blockchain network can include default rules to allocation portions of data as private, permissive, or public. Furthermore, access levels can be defined to determine who can have access to view at least a portion of respective entries stored in the distributed ledger. In another aspect, booking module 330A can generate a unique booking identifier in any format and utilize such booking identifier to generate a schedule reservation.

Turning now to FIG. 3B, illustrated is an example, non-limiting flow diagram 300B of optimizing capacity and scheduling amongst a distributed scheduling and capacity providers corresponding to a personalized medicine supply chain in accordance with one or more implementations described herein. Repetitive description of like elements employed in other embodiments described herein is omitted for sake of brevity.

FIG. 3B illustrates a flow diagram of a system implementation 300B of distributed scheduling engine module 110-1. As an example, distributed scheduling engine module 110-1 can integrate with one or more ordering subsystem 310B such as an Electronic Health Record system or other record system and transmit ordering data to scheduler user interface 320B based on triggering events, querying, calls or data push models. In an aspect, scheduler user interface 320B can be employed by an application (executing on server device(s) 102) that can render on computing device(s) 104 a scheduling interface for users to interact with and execute scheduling operations as well as to facilitate operations executed on a distributed array of stakeholders. Accordingly, scheduler user interface 320B can integrate with scheduling service 330B and display control operations at computing device 104. Furthermore, scheduling service 330B can integrate and aggregate data from disparate systems at runtime.

For instance, in a scenario where a healthcare provider user or a case manager user employs scheduling module 310A (e.g., via scheduler user interface 320B) to schedule a sample collection, manufacturing of a personalized therapeutic, and infuse the therapeutic into the patient, the scheduling module 310A can execute a scheduling service 330B operation that aggregates other capacity service data. Furthermore, capacity module 320A can source, access, curate, and analyze capacity data from various distributed sources and/or respective capacity registries to facilitate scheduling module 310A to execute scheduling service operations based on capacity information.

For instance, scheduling service 330B can transmit a scheduling request to capacity module 320A, which can request, source, access, curate and/or capacity service data from a center of excellence capacity registry 340B. In an implementation, capacity module 320A can source capacity data from distributed collection sites and infusion sites. In another implementation, capacity module 320A can access capacity data published to a registry by distributed collection site data stores or infusion site data stores. Furthermore, the published capacity data can represent unreserved capacity data and reserved capacity data such as in the case of contracted required capacity for a therapy.

In another instance, scheduling service 330B can transmit a scheduling request to capacity module 320A, which can request, source, access, curate and/or capacity service data from a manufacturing and capacity service registry 350B. In an implementation, capacity module 320A can source capacity data from distributed manufacturing and capacity site databases configured for various regions. In another implementation, capacity module 320A can access capacity data published to a registry by distributed manufacturing site data stores and/or databases. Furthermore, the published capacity data can represent unreserved capacity data and reserved capacity data such as in the case of contracted required capacity (e.g., manufacturing capacity) for a therapy.

In another implementation, capacity module 320A can provide a registry configured as a programmable, modular and integrated system for flexibly and securely exchanging data and associated services among one or more electronic devices, systems, and service providers. The registry can be configured as a gateway for one or more devices or data sources, where the registry provides rules for the sources, application programming interfaces (API's) for the service providers, and allows for the underlying data to remain in separate virtual machines that can control and manage the data via separate software stacks via its own servicing modules.

In another instance, scheduling service 330B can transmit a scheduling request to capacity module 320A, which can request, source, access, curate and/or capacity service data from courier capacity service registry 370B. In an implementation, capacity module 320A can source capacity data from one or more courier database configured for various regions. In another implementation, capacity module 320A can access capacity data published to a registry by distributed manufacturing site data stores and/or databases. Furthermore, the published capacity data can represent unreserved capacity data and reserved capacity data such as in the case of contracted required capacity (e.g., courier capacity) for a therapy. In another aspect, scheduling service 330B can access customer configuration and scheduling rules 360B to identify scheduling and capacity rules that impose constraints and structure to the execution of a scheduling operation.

In yet another instance, scheduling service 330B in connection with scheduling module 310A can execute data & prediction service 380B (e.g., using prediction engine module 150-1 to facilitate prediction activities. For instance, data & prediction service 380B (e.g., executed by prediction engine module 150-1) can analyze time interval data and apply prediction models to such data to predict transit times at each leg of a journey through the individualized medicine supply chain. Furthermore, data & prediction service 380B can predict the manufacturing time required to perform a target manufacturing step or entire manufacturing process for a therapeutic product based on historical data and prediction models.

Furthermore, data & prediction service 380B can facilitate real-time schedule optimizations using predictive insights. In another aspect, data & prediction service 380B in connection with scheduling service 330B can assign schedule requests to waitlist queues and over-schedule queues based on predictive insights and rescheduling insights based on rescheduling data. Accordingly, scheduling module 310A can utilize center of excellence capacity service 340B, manufacturing and capacity service 350B, customer configuration and scheduling rules 360B, courier capacity service 370B, and/or data and/or prediction service 380B to generate discrete schedules and/or comprehensive scheduling determinations for the entire process associated with individualized medicine therapy supply chains.

In a non-limiting implementation, center of excellence capacity service 340B, manufacturing and capacity service 350B, and courier capacity service 370B can be connected to wireless sensor networks to provide distributed network and internet access to sensors, controls, and processors that are deeply embedded in equipment, facilities, and the environment. Furthermore, the respective services can provide monitoring and control capability for applications integrated with such sensors. In an aspect, the wireless sensor networks can allow for the sourcing of real-time capacity data, scheduling data, equipment health data, time interval data, and other such data via lower power signal processing, and low power computation. Furthermore, the sensor networks can provide for sensing, local control, and embedded system integrations in structures, materials, and environments.

For instance, manufacturing and capacity service 350B can be communicatively coupled to integrated with sensors embedded within manufacturing machines in order to track and monitor manufacturing scheduling delays, equipment malfunctions related to manufacturing capacity constraints, and inefficiencies in personalized medicine manufacturing processes. In another instance, courier capacity service 370B can be communicatively coupled to smart labels devices and sensors to track package deliveries for therapeutic products (e.g., transported to infusion sites), specimens (e.g., transported to manufacturing facilities), raw materials (e.g., transported to manufacturing sites) and other such items being transported via courier. Furthermore, data and/or prediction service 380B can make use of such sensor data and other data to apply machine learning models and extract insights.

FIG. 3C illustrates an example, non-limiting diagram of an interactive scheduling interface 300C rendered by distributed scheduling system at a client device in association with a personalized medicine supply chain in accordance with one or more implementations described herein. Repetitive description of like elements employed in other embodiments described herein is omitted for sake of brevity.

At FIG. 3C, illustrated is an interactive scheduling interface 300C. At 310C, the interface 300C references the activity to be scheduled and associated data such as apheresis, infusion, manufacturing, location information, date information, and start time information. At 340C, the interface 300C references treatment data, location data, delivery data, and status data. Furthermore, at 320C, illustrated are the current date. Also, at 330C, illustrated are circles around respective dates, where the circles represent an availability of such dates for scheduling a respective activity (e.g., collecting blood). In an aspect, scheduling module 310A can integrate with interactive scheduling interface 300C to render scheduling dates available based on a scheduling model that determines scheduling availabilities based on collection site capacity data, infusion site capacity data, pre-configured or estimated time intervals required to execute a collection operation, pre-configured or estimated time required to transfer a Dewar to a collection site, a pre-configured or estimated time required to transfer materials to a respective manufacturing site(s), a pre-configured or estimated time required to manufacture the final product, a pre-configured or estimated time required to transfer a respective material to an infusion site, and/or a courier capacity to execute a transportation task. In an aspect, scheduling module 310A can execute consolidation activities to consolidate capacities and schedules from disparate systems to render a unified view of dates available for collection.

FIG. 3D illustrates an example, non-limiting flow diagram 300D of scheduling and capacity reservation routines associated with the distributed scheduling system in accordance with one or more implementations described herein. Repetitive description of like elements employed in other embodiments described herein is omitted for sake of brevity.

At FIG. 3D, illustrated is a flow diagram 300D of a two-phase distributed scheduling implementation that mitigates errors in scheduling and enhances scheduling accuracy. An implementation of a two-phase distributes scheduling system enables execution of scheduling a first activity to enable an automated scheduling of down-stream activities within required time frames to enable the therapeutic to maintain its efficacy and half-life. For instance, a two-phase distributed scheduling can execute a scheduling of a blood collection task at a center of excellence based on resource availability (e.g., materials, equipment and room availability, etc.), patients' availability, and skilled labor availability (e.g., nurse schedule) which then executes a down-stream scheduling operation to reserve a capacity slot for manufacturing (e.g., manufacturing site capacity to manufacture the therapeutic from the patient drawn blood) and generate a schedule reservation for infusion of the therapeutic within medically prescribed time windows and in accordance with scheduling and capacity availability of down-stream phases.

As a non-limiting example, at 310D the scheduling module 310A can receive scheduling request input from a scheduling query initiated by client distributed scheduling module 195 of computing device(s) 104. Furthermore, scheduler module 310A transmits date options to client distributed scheduling module 195 for propagation as circled dates at interactive scheduling interface 300C. The scheduler module 310A receives date selection input from client distributed scheduling module 195. Furthermore, at 320D, scheduler module 310A generates a schedule record and assigns a pending state status flag to the schedule record. Furthermore, scheduler module 310A transmits the schedule record to booking module 330A for storage of the schedule record in persistent storage.

In another aspect, scheduler module 310A can generate a pending reservation for recognition and analysis by a respective distributed database associated with the site for which the schedule pertains. For instance, scheduler module 310A can ping or call a center of excellence capacity service 340B to generate a pending reservation (e.g., see reference numeral 320D) at a target collection and infusion site database assigned to the site in which the collection is scheduled to occur. Furthermore, scheduler module 310A can ping or call a first downstream service provider site such as manufacturing and capacity service 350B to generate a pending reservation (e.g., see reference numeral 330D) at a target manufacturing site database assigned to the site in which manufacturing or intermediate manufacturing is scheduled to occur. In another aspect, scheduler module 310A can subsequently or simultaneously ping or call a second downstream service provider site such as a courier capacity service 370B to generate a pending reservation (e.g., see reference numeral 330D) at a target courier database assigned to the courier in which transportation is scheduled to occur within define time intervals and such pending reservation can be generated for each leg of the process (e.g., courier from collection site to manufacturing site, courier from manufacturing site to infusion site, etc.).

At reference numeral 360D, booking module 330A can analyze the pending request against rules, limitations, restrictions, capacity constraints, personnel schedules, site specific data and other scheduling requests to generate a confirmation or denial of the pending request. Furthermore, booking module 330A can transmit a success or failure response based on the ability to confirm or deny a pending request inquiry. In another aspect, booking module 330A can assign a unique booking identifier, in any range of formats, to the respective service (e.g., 340B, 350B, 370B, etc.) and scheduler module 310A. Furthermore, scheduling module 310A can couple the booking identifier to the scheduled reservation. For instance, a scheduled reservation can map to an ordering of steps and corresponding reservation status with each step to represent a journey map throughout the individualized medicine production chain or supply chain. As an example, a collection step can correspond to a collection reservation identifier corresponding to namespaced values such as systemA://reservation/<id of the reservation in system A, and state pending.

A similar identifier and mapping operation can be executed for other activities such as manufacturing, infusion, cryogenic storage, transportation, and other such activities. In an aspect, booking module 330A can transmit a successful (e.g., reference numeral 360D) identifier or a failure identifier to scheduling module 310A. Furthermore, if scheduling module 310A determines approval of the pending reservation status is required (e.g., see reference numeral 350D), scheduling module 310A can request approval (e.g., see reference numeral 340D) and verification of the successful generation of the pending reservation to a controller application (e.g., a second user operates such application) to either approve (e.g., see reference numeral 380D) and finalize the status of pending reservation or deny the pending reservation. In the event that, the pending reservation is denied, then the controller application notifies the denial to scheduling module 310A and scheduling module 310A cancels the reservation (e.g., see reference numeral 370D.

In another aspect, if scheduling module 310A determines that an approval is not required from the controller application, then booking module 330A can confirm the reservation (see reference numeral 390D). Furthermore, upon scheduling module 310A determining a successful pending reservation occurred (e.g., see reference numeral 392D), then the schedule is confirmed (e.g., see reference numeral 394D) or in the event the schedule is denied, then scheduling module 310A cancels the reservation (e.g., see reference numeral 370D). Also, at any time scheduling module 310A can be configured to retry generating pending reservation for a predefined number of attempts before a schedule is cancelled via executing a rollback operation to return the scheduling application to a previous version of the reservation statement (e.g., undo the reservation, or remove the previous reservation statement).

Furthermore, the scheduling module 310A can attempt to call or ping respective services to re-attempt generation of a pending reservation. In an aspect, such rollback operations can be implemented based on a time scheduled mechanism such that each service is configured to cancel a generated pending reservation after a threshold time interval has passed and no confirmation has been generated. Furthermore, a time interval-based rollback operation can be implemented across a distributed array of scheduling subsystems by scheduling module 310A such that all pending reservations along an up-stream and down-stream process can be successfully rolled back in an instance. In an aspect, upon approval of a pending reservation, if approval is required (e.g., as per rule configurations), then scheduling module 310A can confirm a reservation based on a call, request or ping of each respective service over another instance using the booking identifier returned by the service (e.g., using booking module 330A) at the time the original pending reservation was generated.

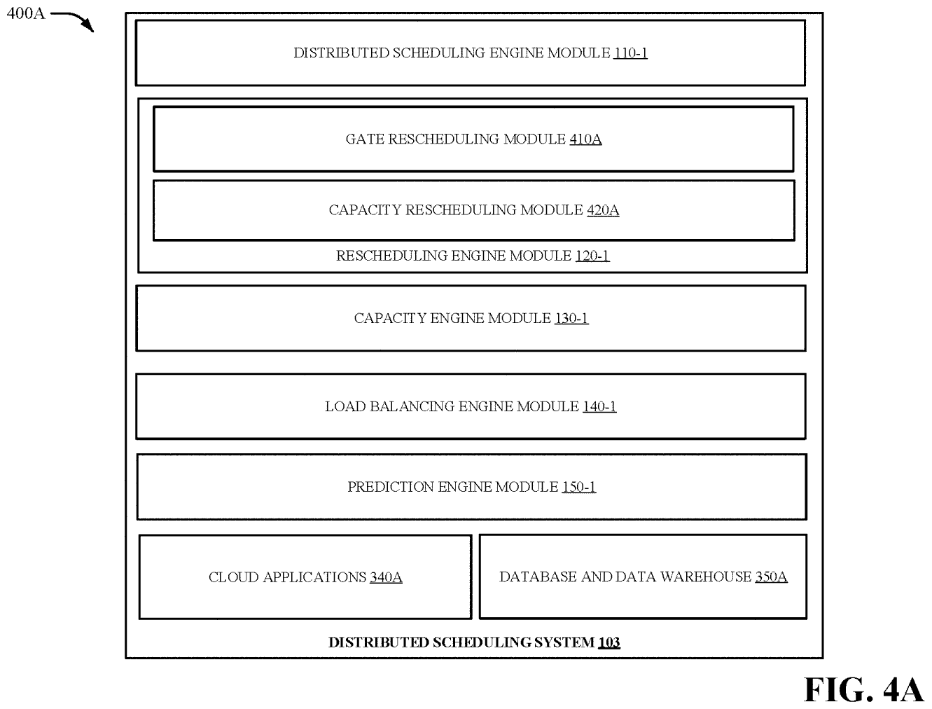

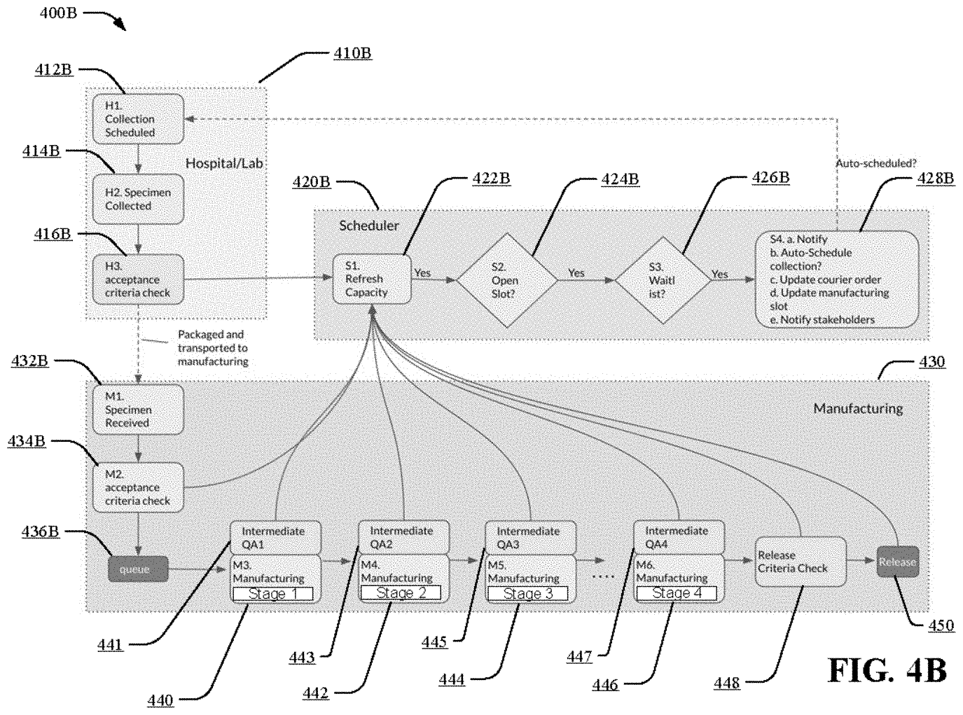

FIG. 4A illustrates an example, non-limiting distributed scheduling system 400A and an example rescheduling engine module in accordance with one or more implementations described herein. Repetitive description of like elements employed in other embodiments described herein is omitted for sake of brevity.

In an implementation, distributed scheduling system 103 can employ rescheduling engine module 120-1 that executes gate rescheduling module 410A and capacity rescheduling module 420A. In accordance with the individualized medicine therapy production chain and supply chain, a series of steps result in the generation of an individualized therapeutic product for use by a target patient. At each step of the process a rescheduling event can be executed by reschedule engine module 120-1. Rescheduling can occur for a variety of reasons such as schedule conflicts of patients, weather/traffic delays for couriers, manufacturing delays due to equipment failures, capacity constraint related rescheduling, material limitations relate to rescheduling, and other such reasons for rescheduling.

In an implementation, rescheduling engine module 120-1 can employ gate rescheduling module 410A to generate quality gates that can trigger a hold on downstream scheduling events based on the failure to satisfy predefined gate criteria. Furthermore, in an aspect, the gate rescheduling module 410A can generate, source and curate data related to a process and apply an estimation model to such gate data to estimate or re-estimate the remaining time required to complete the generation of the final therapeutic product as well as time required for such product to arrive at the infusion site.

FIG. 4B illustrates a flow diagram of an example, non-limiting quality control-based scheduling and rescheduling operations as well as estimation operations associated with the distributed scheduling system in accordance with one or more implementations described herein. Repetitive description of like elements employed in other embodiments described herein is omitted for sake of brevity.

At diagram 400B, presented is a depiction of estimation techniques and illustrations of gate mechanisms employed by rescheduling engine module 120-1. In an aspect, reference numeral 410B is a scheduling service for a hospital or lab in relation to a specimen collection activity. At reference numeral 420B is a distributed scheduling engine module implementation (also referred to as distributed scheduling engine module 110-1). At reference numeral 430 is a manufacturing scheduling service for a manufacturing site. In an aspect, between each service and distributed scheduling engine module 420B are gates implemented using gate rescheduling module 410A. Each gate represents a series of quality checks that set forth acceptance criteria for scheduling and operations to proceed to a subsequent step in the individualized medicine supply chain. Furthermore, in an aspect, if quality check criteria are unsatisfied or a new estimate is generated for time intervals to complete an operation, then such quality check data is transmitted to scheduling module 310A.

Accordingly, scheduling module 310A in connection with capacity rescheduling module 420A can utilize updated capacity information from source capacity service databases (e.g., courier systems, manufacturing execution systems, infusion and collection capacity systems. In some instances, scheduling module 310A can be the system of record for some capacity data related to capacity service systems. In the event new capacity are determined to be available, scheduling module 310A can notify systems configured as relevant of such capacity slot availability. Furthermore, in an aspect, scheduling module 310A in connection with gate rescheduling module 410A can access and query a waitlist database to identify whether an available slot is immediately available or waitlisted against a queue of waitlisted users for scheduling. In the event, there is no waitlist queue, then scheduling module 310A can auto-schedule (based on such configuration) to automatically schedule a user in the new available scheduling slot. Furthermore, scheduling module 310A can notify all respective service databases and client devices of the occurrence of such rescheduling event.

As an example, reference numeral 410B identifies a hospital or lab service that can perform a scheduled collection 412B, a collection of the specimen 414B, and either satisfies or fails to satisfy acceptance criteria check 416B. Upon satisfaction of acceptance criteria, such notification of acceptance is transmitted by the hospital lab service at reference numeral 410B to distributed scheduling engine module 420B. Furthermore, a collected sample can be packaged and transported to a manufacturing site 430 from a hospital or lab site 410B. In another aspect, the manufacturing service 430 can determine a receipt of the specimen 432B. Furthermore, a quality control gate can determine whether the received specimen satisfies acceptance criteria (e.g., see reference numeral 434B). At queue 436B, a set of specimens awaiting manufacturing can be queued in a waiting line.

In an aspect, at intermediate QA1 441, intermediate QA2, Intermediate QA3, and Intermediate QA4, gate rescheduling module 410A has established gates at intermediary stages of manufacturing an individualized therapeutic medicine. For instance, therapeutic manufacturing stage one 440 is paired with intermediate quality acceptance criteria 441, manufacturing stage two 442 is paired with intermediate quality acceptance criteria 443, manufacturing stage three 444 is paired with intermediate quality acceptance criteria 445, manufacturing stage four 446 is paired with intermediate quality acceptance criteria 447. Furthermore, upon acceptance of the quality acceptance criteria a next phase of scheduling can proceed.

In another aspect, after the therapeutic has been checked for acceptance of release criteria (e.g., using gate rescheduling module 410A), the therapeutic product can be released (e.g., see reference numeral 450) from manufacturing to the next scheduled activity (e.g., courier transportation to infusion center). In another aspect, distributed scheduling engine module 420B can access real-time manufacturing capacity information from relevance stages of manufacturing occurring (e.g., see reference numeral 434B, 440, 442, 444, 446, and 448). As such, based on the capacity data, distributed scheduling engine module 420B can determine whether a manufacturing capacity slot is available (e.g., see reference numeral 424B) for a scheduling or rescheduling operation.

In the event an available slot is present, capacity rescheduling module 420A can reschedule a waitlist 426B user or product to execute a respective operation (e.g., commence a manufacturing operation). In the event, such rescheduling occurs, then rescheduling engine module 120-1 can notify other services (e.g., manufacturing service, collection service, etc.), auto-schedule various operations including downstream activities (e.g., auto-schedule a specimen collection), transmit updates to courier orders (e.g., adjust a time interval for a courier pick-up and drop-off), update manufacturing slot availability from open to unavailable, and notify relevant stakeholder systems of the re-scheduling operation.

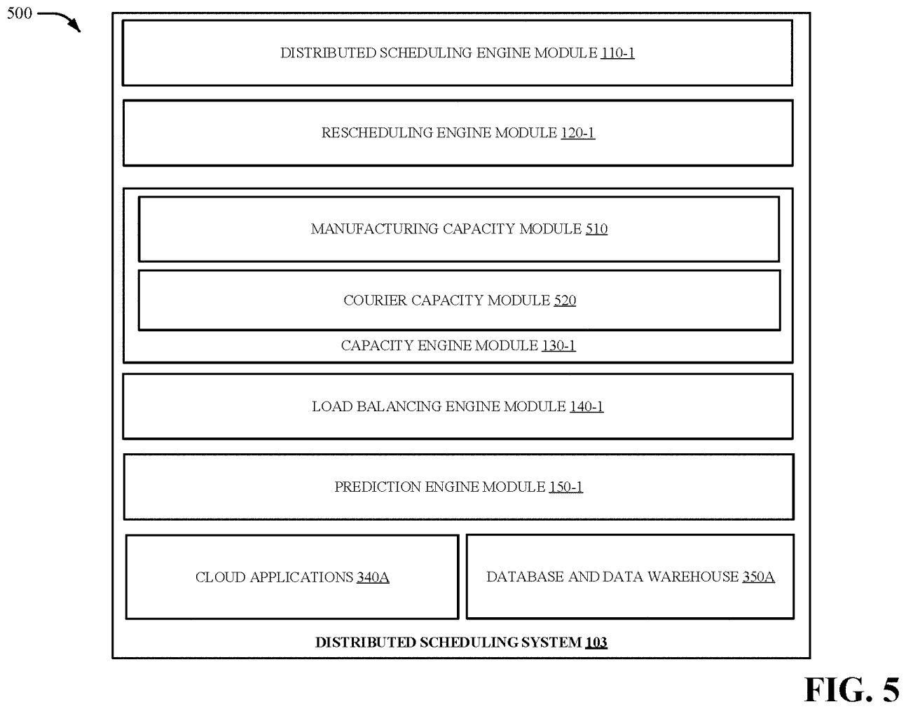

FIG. 5 illustrates an example, non-limiting distributed scheduling system 500 and an example capacity engine module in accordance with one or more implementations described herein. Repetitive description of like elements employed in other embodiments described herein is omitted for sake of brevity.

Turning now to distributed scheduling system 500 which provides an implementation of distributed scheduling system 103 that includes capacity engine module 130-1 that executes manufacturing capacity module 510 and courier capacity module 520. In an aspect, manufacturing capacity module 510 can optimize manufacturing operations across several facilities based on identifying available manufacturing slots at distributed manufacturing sites and mapping schedules of therapies requiring production to an optimal available manufacturing site based on a set of relevancy factors (e.g., transportation distance, available capacity, etc.). In another aspect, manufacturing capacity module 510 can execute a determination model that determines a manufacturing facility across distributed manufacturing sites that can manufacture a particular therapy on a given date.

In an instance, the determination model can make a determination of manufacturing facility based on several respective parameters that can be tuned. As such, the configurable parameters can include whether the facility is a primary facility versus a backup facility, available capacity, proximity of a manufacturing site to a departure site (e.g., collection facility, cryogenic storage facility, etc.), day of the week, load balance considerations across sites, fastest predicted site for manufacturing, fasted predicted transportation time between sites, and other such parameters. In an instance, the parameter of closes site versus shortest travel time to a manufacturing site refers to a manufacturing site having an estimated time to transfer a material from a collection site to a manufacturing site and from a manufacturing site to an infusion site.

In an aspect, the determination model can employ a neural network model to propagate layers of determinations to select optimal sites and provide estimated transit times. In an aspect, a transit time may not merely be proportional to a distance between sites but rather may need to consider other factors specific to the mode of transportation (e.g., air, car, rail, etc.), traffic conditions, weather conditions, requirements of the therapeutic or specimen, and other such considerations. With respect to parameters that predict the fastest manufacturing and transportation timings, such predictions can be based on prediction models applied to historical data to estimate future predictions. In another aspect, other parameters can take into account variance across manufacturing sites such as type of machines, age of equipment, quality control processes, standard operating procedures, and other such variables. Due to the non-standard nature of such variables, such parameters can be incorporated into determination models via tagging operations, rule-based implementations, and other such mechanisms.

In some instances, a therapy manufacturer commissioned to manufacture an individualized medicine therapeutic product may contract multiple couriers to service different or the same routes. As such, capacity engine module 130-1 can employ courier capacity module 520 configured to determine an optimal courier for a specified route based on deployment of a smart schedule model. In an aspect, courier capacity module 520 can deploy an algorithmic model that considers various parameters such as whether a courier is a primary or backup courier, predicted best routes for transportation, load balance across couriers, and cost considerations.

In an aspect, the parameter related to predicting the best courier for a particular route is based on historical data. Furthermore, such predictions can be based on variables such as weather conditions, a particular port of entry, exit port, delay details and other such considerations. For example, a respective courier may execute optimally under certain conditions or between particular locations, or via a respective mode of transportation. In an aspect, courier capacity module 520 can employ machine learning techniques to extract learnings related to such variables and parameters and tweak prediction models to better predict optimal couriers for particular routes. In other aspects, courier capacity module 520 can employ parameters that based determinations, in part on, cost considerations such as courier rates, courier reliability, courier speed of transportation, courier cost for slower transporters that still deliver within required time intervals (e.g., cost benefit analysis), and other such considerations. Furthermore, courier capacity module 520 can execute operations in connection with smart scheduling techniques to automatically select a courier to optimize a cost of delivery.

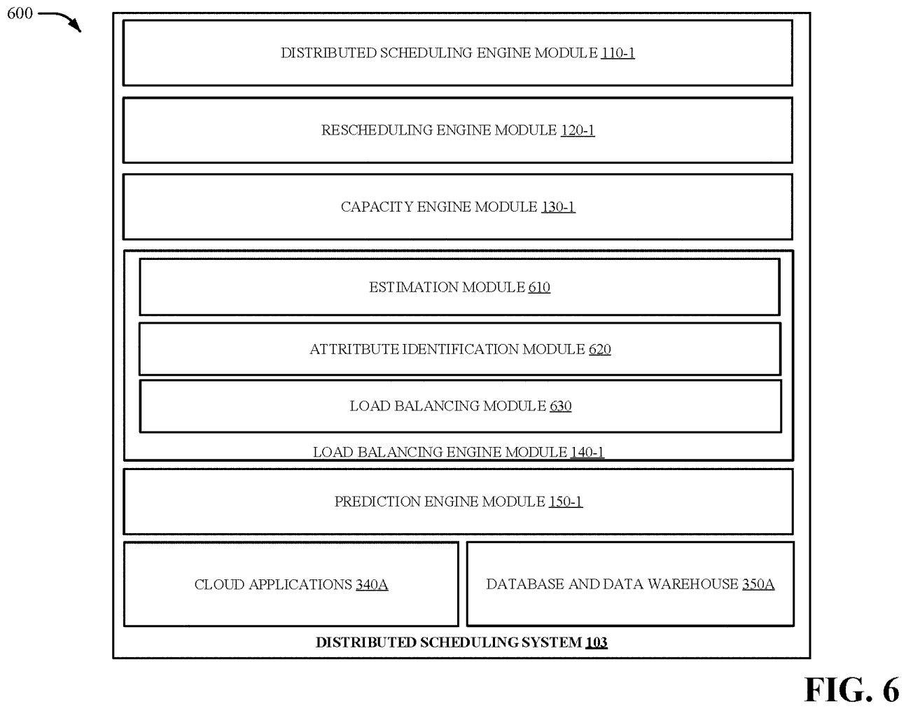

FIG. 6 illustrates an example, non-limiting distributed scheduling system 600 and an example load balancing engine module in accordance with one or more implementations described herein. Repetitive description of like elements employed in other embodiments described herein is omitted for sake of brevity.

In an aspect, distributed scheduling system 103 in implementation 600 can employ load balancing engine module 140-1 to balance capacity loads across a distributed array of manufacturing sites. As such, load balancing engine module 140-1 in connection with distributed scheduling engine module 110-1 can balance scheduling activities to satisfy available capacity slots at each respective site for a particular date based on estimations of time intervals corresponding to each capacity slot (e.g., time interval required to complete each intermediary manufacturing step) for manufacturing activities on particular dates and corresponding capacity during such time durations. Accordingly, load balancing engine module 140-1 can accomplish scheduling and capacity load balancing operations by executing estimation module 610, attribute identification module 620, and load balancing module 630.

In an aspect, estimation module 610 can estimate a time to manufacture a particular therapy based on therapy specific manufacturing parameters such as cytometry data related to a patients collected specimen, patient biomarker data, and length of time to manufacture a therapy for a particular patient based on such cytometry data and biomarker data. Accordingly, such estimation data can be iteratively utilized in a feedback loop to extract insights, enhance machine learning decisions, and identify patterns related to manufacturing as pertains to particular therapies. Also, such estimation data can be utilized by scheduling module 330A to adjust scheduling suggestions based on patient specific insights. In another aspect, estimation module 610 can estimate a time of arrival of a therapeutic product at a manufacturing site using long short-term memory (LSTM) network prediction techniques. For instance, an LSTM network can utilize recurrent neural network (RNN) techniques to learn long-term dependencies to provide accurate predictions related to delays for delivery times and manufacturing times. In an aspect, the RNN LSTM architecture can a Back Propagation Through Time technique to train algorithms using historical input feature values (e.g., one week of scheduling values) to update weights in recurrent neural networks to predict the behavior of the most influential features of the scheduling model and capacity model. Furthermore, in an aspect, the accuracy of the model predictions can be evaluated against subsequent scheduling data.

In another aspect, estimation module 610 can integrate with manufacturing equipment systems to enable real-time data analysis and utilization for estimation activities. Furthermore, such system integrations can ensure machine parameters can be considered for routing or re-scheduling therapy manufacture in real time. For instance, if a manufacturing machine malfunctions or is unavailable unexpectedly (e.g., due to lack of qualified resources), estimation module 610 can account for such unavailability or malfunction and determine an estimated impact on time of manufacture. Furthermore, estimation module 610 can communicate with re-scheduling modules to immediately execute manufacturing re-scheduling operations if available.

In another aspect, an attribute module 620 can be employed to evaluate attributes specific to particular manufacturing facilities for determination of load balancing of capacities. In another aspect, load balancing engine module 140-1 can employ load balancing module 630 configured to facilitate load balancing based on load factor considerations. For instance, load balancing module 630 can apply a load factor that indicates resource availability to carry out demands associated with a respective capacity. For example, load balancing module 630 may assign a load factor of 0.75 to a model determining a manufacturing load capacity, where the load factor indicates that the facility cannot execute manufacturing jobs beyond 75% of the respective facilities capacity.

In another implementation, load balancing engine module 140-1 can also balance route requests to different server instances associated scheduling operations, rescheduling, operations, and manufacturing operations. Furthermore, load balancing engine module 140-1 can allow for the scaling of data access operations, by supporting routing operations of different server instances across distributed scheduling sources. As such, load balancing engine module 140-1 may distribute scheduling requests among different server instances in a distributed manner that reduces processing inefficiencies.

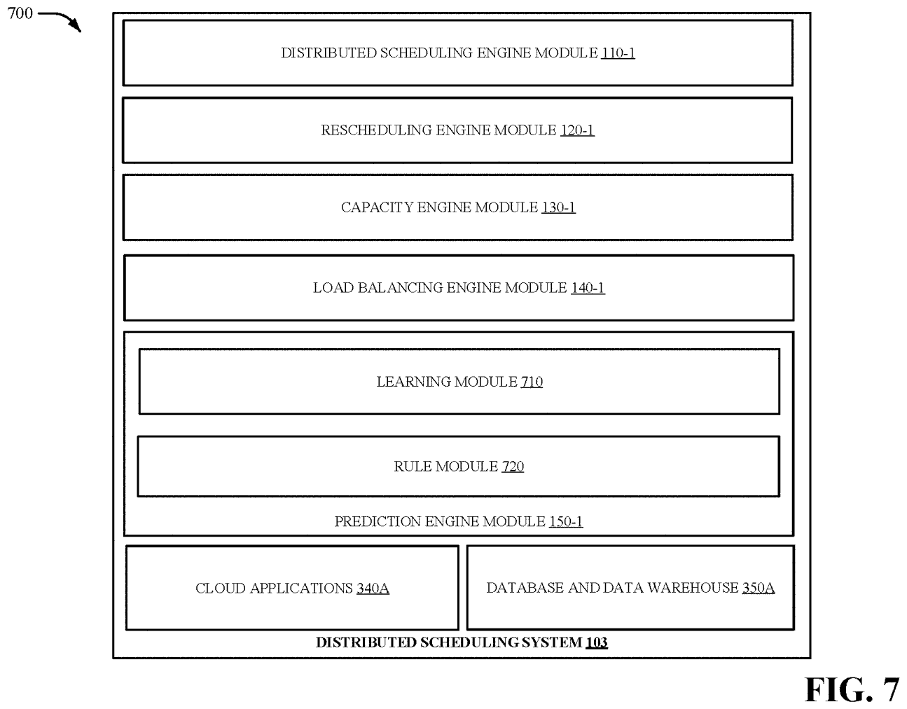

FIG. 7 illustrates an example, non-limiting distributed scheduling system 700 and an example prediction engine module in accordance with one or more implementations described herein. Repetitive description of like elements employed in other embodiments described herein is omitted for sake of brevity.

In an aspect, distributed scheduling system 700 can employ prediction engine module 150-1 to execute learning module 710 and rule module 720 to facilitate predictions related to scheduling and procurement of therapies. In an aspect, learning module 710 can be configured to predict a patient likelihood to reschedule and an impact of a patient rescheduling. For instance, learning module 720 can deploy a learning model to extract insights from data related to past patient appointments, other patient's conditions at the time of canceled appointments, and determine a likelihood of cancellation based on a comparison of a patient's profile to other patient profiles for a particular site.

In another aspect, rule module 720 in connection with learning module 710 can apply machine learning models against patient data to predict time duration required to plan and perform in real-time tasks such as collecting patient material, transporting collected material to manufacturing sites including intermediary legs of transportation, accounting for flight delays and other travel related factors, manufacturing of a final product (e.g., using integrations from manufacturing systems), transportation of final products back to the infusion site including all intermediate legs of the journey, and time predictions to transport a package from one location to the next or from one manufacturing step to the next manufacturing step (e.g., based on smart label detection).

In an aspect, these predictions can impact manufacturing slot availabilities and timing of downstream activities such as infusion schedules. Because learning module 710 and rule module 720 can execute prediction operations across multiple manufacturing sites and couriers, such predictions can prepare the individualized medicine therapy supply chain to have backup capacities and scheduling options lined up. Furthermore, such backups can allow for the scaling of cell and gene therapy production and the minimization of bottlenecks and single points of failure in production processes.

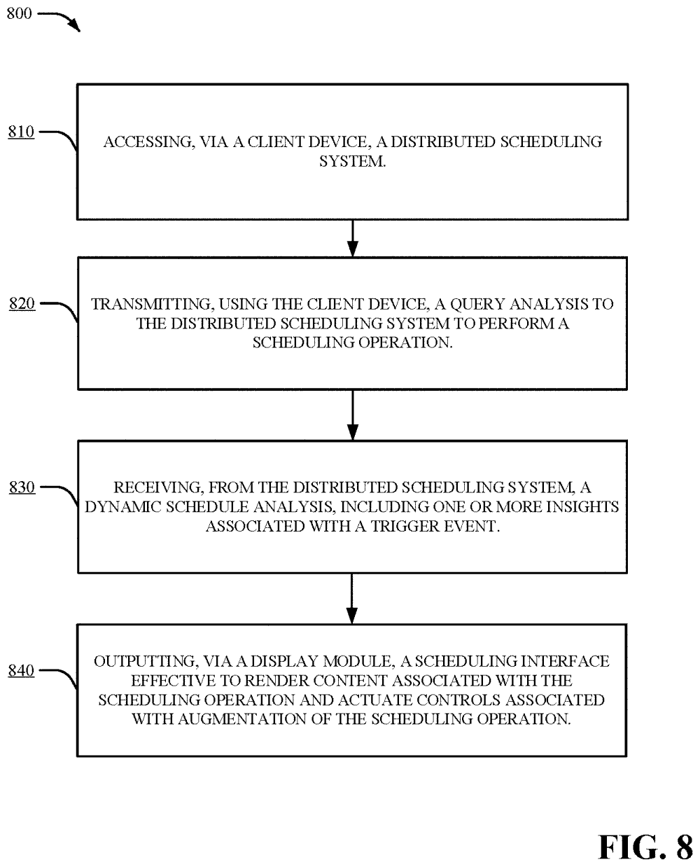

FIG. 8 illustrates a flow diagram of accessing, via a client device, a distributed scheduling system in accordance with one or more implementations described herein. Repetitive description of like elements employed in other embodiments described herein is omitted for sake of brevity.

At reference numeral 810, a client device accesses a distributed scheduling system. At reference numeral 820, the client device transmits a query analysis to the distributed scheduling system to perform a scheduling operation. At reference numeral 830, the distributed scheduling system receives a dynamic schedule analysis including one or more insights associated with a trigger event. At reference numeral 840, a display module outputs a scheduling interface effective to render content associated with the scheduling operation and actuate controls associated with augmentation of the scheduling operation.

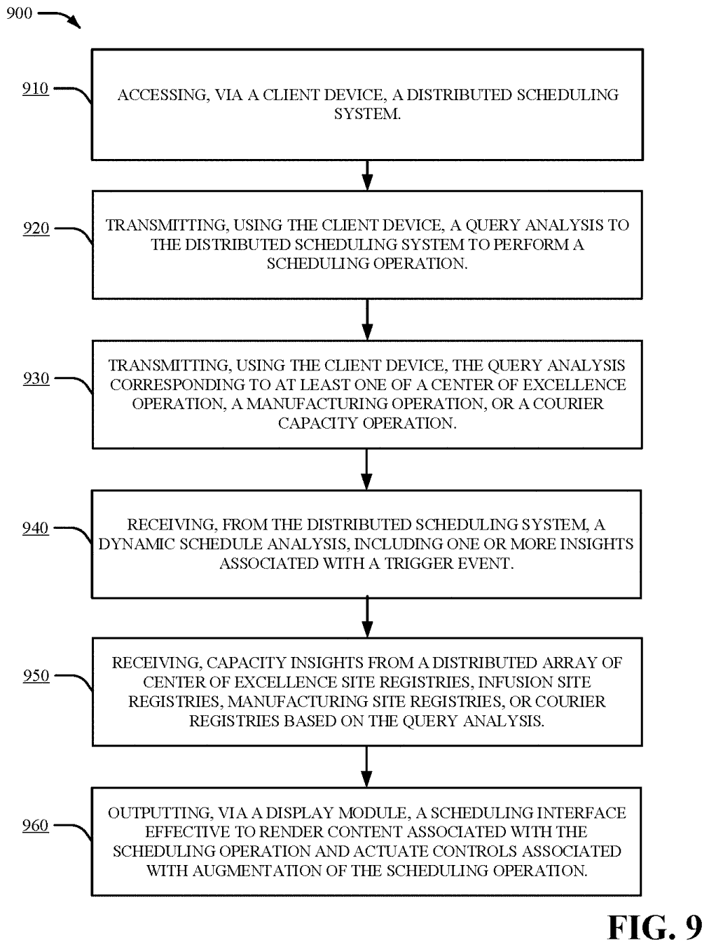

FIG. 9 illustrates a flow diagram of querying, via a client device, a distributed scheduling system in accordance with one or more implementations described herein. Repetitive description of like elements employed in other embodiments described herein is omitted for sake of brevity.

At reference numeral 910, a client device accesses a distributed scheduling system. At reference numeral 920, the client device transmits a query analysis to the distributed scheduling system to perform a scheduling operation. At reference numeral 930, the client device transmits the query analysis corresponding to at least one of a center of excellence operation, a manufacturing operation, or a courier capacity operation. At reference numeral 940, the distributed scheduling system receives a dynamic schedule analysis including one or more insights associated with a trigger event. At reference numeral 950, the client device receives capacity insights from a distributed array of center of excellence site registries, infusion site registries, manufacturing site registries, or courier site registries based on the query analysis. At reference numeral 960, a display module outputs a scheduling interface effective to render content associated with the scheduling operation and actuate controls associated with augmentation of the scheduling operation.

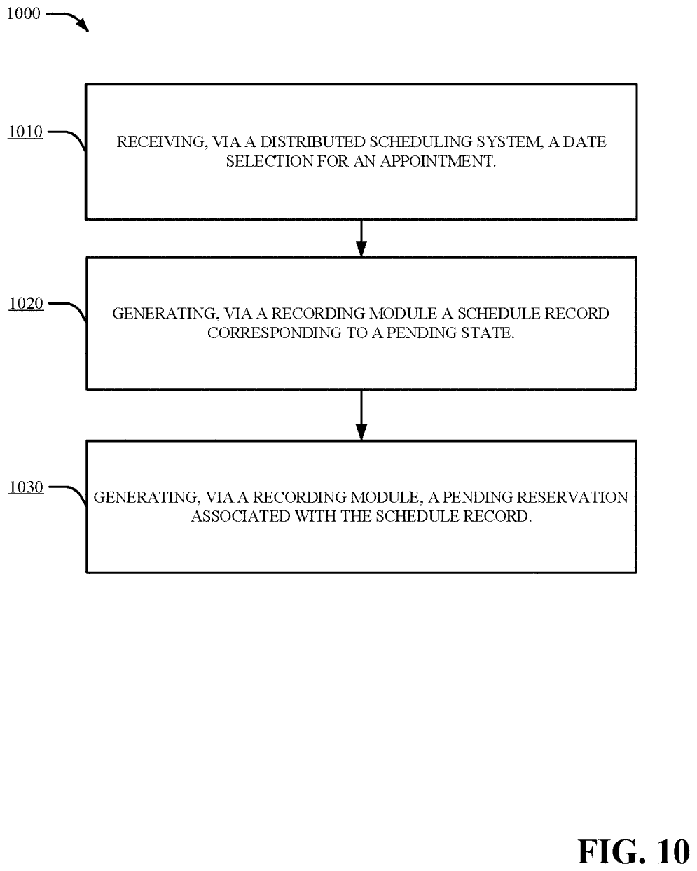

FIG. 10 illustrates a flow diagram of generating, via a distributed scheduling system a schedule record in accordance with one or more implementations described herein. Repetitive description of like elements employed in other embodiments described herein is omitted for sake of brevity.

At reference numeral 1010, a distributed scheduling system receives a date selection for an appointment. At reference numeral 1020, a recording module generates a schedule record corresponding to a pending state. At reference numeral 1030, a pending reservation is generated associated with the schedule record.

What is claimed is:

1. A system comprising:
one or more processors; and
one or more storage devices comprising processor executable instructions that, responsive to execution by the one or more processors, cause the system to perform operations comprising:
curating scheduling data from a distributed set of data stores corresponding to at least one of a patient database, a collection site, a courier data store, or an infusion site;
orchestrating a scheduling alignment between a set of sites corresponding to the distributed set of data stores based on predefined rules and predefined dependencies; and
determining a scheduling availability and a capacity for executing scheduled tasks based on the scheduling alignment.

2. The system of claim 1, wherein the operations further comprise:
analyzing events corresponding to the scheduling availability and the capacity for executing scheduled tasks; and
predicting a likelihood of a rescheduling event occurring based on the analyzing, wherein the event occurring is at least one of a patient rescheduling, an addition of one or more patients to a scheduling waitlist, a first time duration for collection of a material, a second time duration to transport collected material to a manufacturing site, a third time duration to transport a therapeutic product to an infusion site, a batching of multiple orders for pickup.

3. The system of claim 1, wherein the operations further comprise:
curating a set of capacity data from one or more distributed data sources, wherein a first subset of the set of capacity data represents cache capacity time stamp information and a second subset of the set of capacity data represents cache refresh time interval information; and
generating, based on the set of capacity data, a logical query that extracts target capacity data of the set of capacity data from the one or more distributed data sources based on a scope of the logical query.

4. The system of claim 3, wherein the one or more distributed data sources are at least one of a collection site system, an infusion site system, a manufacturing site system, or a courier system.

5. The system of claim 1, wherein the operations further comprise:
receiving, via a distributed scheduling module, a scheduling query associated with a set of personalized medicine supply chain systems comprising at least one of a collection site system, an infusion site system, a manufacturing site system or a courier system;
analyzing, via the distributed scheduling module, a set of capacity data corresponding to the collection site system, the infusion site system, the manufacturing site system, or the courier system, wherein the set of capacity data comprises at least one of collection capacity data, infusion capacity data, estimated collection time, estimated Dewar transfer time, estimated material transit time to manufacturer, manufacturing capacity data, estimated manufacture time, transit infusion time data or courier capacity data.

6. The system of claim 2, wherein the operations further comprise:
predicting a likelihood of occurrence of a rescheduling event or a scheduling cancelation on a set of distributed schedules associated with personalized medicine supply chain events;
determining, based on the predicted likelihood, an impact of the rescheduling event or the scheduling cancelation on the set of distributed schedules; and
modifying the set of distributed schedules based on the predicted likelihood corresponding to a likelihood rank that fall within a predefined threshold associated with a scheduling event occurring.

7. The system of claim, 1, wherein the operations further comprise:
predicting a time interval to execute one or more operation corresponding to personalized medicine supply chain events, wherein the one or more operation comprises at least one of a material collections, material transportation, manufacture a therapy, transportation of a therapeutic product; time for delivery of the therapeutic product.

8. The system of claim 1, wherein the operations further comprise:
generating a set of capacity data from a set of distributed capacity scheduling data sources;
storing a subset of generated capacity data in a cache corresponding to a respective scheduling data source;
ordering the subset of generated capacity data based on recency of the subset of generated capacity data; and
refreshing the cache based on a recurring time interval or occurrence of a triggering event by the respective scheduling data source.

9. The system of claim 8, wherein the operations further comprise:
generating the set of capacity data from set of registries comprising a center of excellence registry, and manufacturing registry, and a courier capacity registry.

10. The system of claim 1, wherein the operations further comprise:
outputting the scheduling availability via an interface module effective to:
render a calendar that denotes dates configured to allow for collection based on a set of time conditions and capacity conditions, wherein the capacity conditions comprise at least one of a collection site capacity, courier capacity, manufacturing site capacity or an infusion site capacity, and wherein the set of time conditions comprise at least one of a time required for collection, a manufacturing time, or a transit infusion time.

11. A system comprising:
a processing system that implements a distributed scheduling system comprising:
a scheduling engine module configured to:
create a schedule record corresponding to a pending state;
stores the schedule record in a persistent storage device;
extract a pending reservation from a collection site database, an infusion site database, and a manufacturing site database; and
generate one or more courier reservation corresponding to one or more leg of a scheduled routine;
confirming a successful reservation or canceled reservation based on receipt of approvals or denials from a client schedule module of a computing device; and
an identification engine module configured to:
assign a booking identifier to the successful reservation for storage within at least one of the following distributed databases: collection site database, the infusion site database, or the manufacturing site database; and
curate the booking identifier with a set of booking identifiers at a reservation database within an abstraction layer communicatively connected to the distributed databases.

13. A method comprising:
accessing, via a client device, a distributed scheduling system;
transmitting, using the client device, a query analysis to the distributed scheduling system to perform a scheduling operation;
receiving, from the distributed scheduling system, a dynamic schedule analysis, including one or more insights associated with the query analysis; and
outputting, via a display module, a scheduling interface effective to:
render content associated with the scheduling operation; and
actuating controls associated with augmentation of the scheduling operation.

14. The method of claim 13, further comprising:
transmitting, using the client device, the query analysis associated with a predicted impact of one or more rescheduling event; and
receiving, using the client device, the predicted impact of the one or more rescheduling event based on distributed schedules of the distributed scheduling system in connection with manufacturing an individualized medicine product.

15. The method of claim 13, further comprising:
transmitting, using the client device, the query analysis corresponding to at least one of a center of excellence operation, a manufacturing operation, or a courier capacity operation;
receiving, capacity insights from a distributed array of center of excellence site registries, infusion site registries, manufacturing site registries, or courier site registries based on the query analysis.

16. The method of claim 13, further comprising:
transmitting, using the client device, the query analysis to the distributed scheduling system to perform a scheduling operation; and
receiving, using the client device, predicted delivery time events, predicted delay events, and predicted scheduling impacts from delays based on neural network learnings applied to distributed scheduling databases.
